(19) United States
(12) Patent Application Publication
(10) Pub. No.: US 2023/0329813 A1
(43) Pub. Date: Oct. 19, 2023

(54) Systems And Methods For Guided Placement Of A Robotic Manipulator

(71) Applicant: MAKO Surgical Corp., Weston, FL (US)

(72) Inventors: Abdullah Zafar Abbasi, San Diego, CA (US); Amin Eldemerdash, San Diego, CA (US); Steven Polomski, Union City, NJ (US); Zhifu You, Palmetto Bay, FL (US); Jason Karl Otto, Sioux Falls, SD (US)

(73) Assignee: MAKO Surgical Corp., Weston, FL (US)

(21) Appl. No.: 18/135,280

(22) Filed: Apr. 17, 2023

Related U.S. Application Data
(60) Provisional application No. 63/332,024, filed on Apr. 18, 2022.

(51) Int. Cl. A61B 34/32; A61B 34/20; A61B 34/00

(52) U.S. Cl. CPC A61B 34/32; A61B 34/20; A61B 34/25; A61B 34/74; A61B 2034/305

(57) ABSTRACT

Systems, methods, and guidance techniques for guiding placement of a robotic manipulator relative to an anatomy. A localizer tracks the robotic manipulator and the anatomy. Controller(s) obtain workspace parameters of the robotic manipulator and capture, from the localizer, a current state of the robotic manipulator relative to the anatomy. The controller(s) capture, from the localizer, states of the anatomy in response to movement of the anatomy according to a prescribed manner or a predetermined manner and determine operative parameters of the anatomy based on the captured states. The controller(s) compare the workspace parameters to the operative parameters to determine a desired state for the robotic manipulator relative to the anatomy, whereby the workspace parameters of the robotic manipulator have an acceptable relationship with respect to the operative parameters of the anatomy. The controller(s) guide placement of the robotic manipulator from the current state to the desired state.

### CROSS-REFERENCE TO RELATED APPLICATIONS

The subject application claims priority to, and all the benefits of U.S. Provisional Pat. Application No. 63/332,024, filed on Apr. 18, 2022, the entire contents of which are hereby incorporated by reference.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

Traditionally, manipulators have been arranged according to general written instructions associated with a type of procedure being performed. Such instructions, for instance, may give approximated direction on where or how to place the manipulator, e.g., “place robotic arm above the patient and parallel to the surgical table”. The surgical staff then manually arranges the manipulator according to these instructions. In some instances, the patient is manually moved to an approximate region of the manipulator after the manipulator is placed. Many times, surgical staff visually approximate the relationship between the patient and manipulator based on the written directions.

Such prior guidance systems have many shortcomings. For instance, the surgical staff must still manually arrange the manipulator. Consequently, human error remains a limitation. Also, the location at which the manipulator should be located is often defined based on an approximated acceptable range, e.g., 2-4 feet from the surgical table. Therefore, conventional guided arrangement of the manipulator to a location may be acceptable, but sub-optimal. Furthermore, the characteristics of the anatomy, such as the length or height of a limb and/or the range of motion of a joint, will vary from patient to patient. The described approximated placement of the manipulator does not consider these patient-specific variables. Additionally, the working boundary of the robotic manipulator, e.g., the limits of where the robotic arm can reach, can also change throughout the surgical procedure. For instance, the manipulator may be constrained to three degrees-of-freedom in one step of the procedure and may be constrained to four degrees-of-freedom in another step of the procedure. The described approximated placement of the manipulator does not consider these robot-specific variables. Moreover, the specifics of a surgical plan or procedure can change throughout surgery. For instance, a total hip procedure may require several different poses of the manipulator and/or a surgeon may need to tilt a patient’s knee during a total knee procedure. The described approximated placement of the manipulator does not consider these procedure-specific variables.

As a result, conventional guidance systems are still susceptible to the risks for human error and improper or sub-optimal positioning of the manipulator due to their inability to adapt to patient, robot, and/or procedure specific variables, or changes thereof. A condition arising from these variables may require unexpected halting of the surgical procedure and rearrangement of the robotic manipulator to another position, which can cause inconvenience to staff and interruption and delay to the surgical procedure.

### SUMMARY

This Summary introduces a selection of concepts in a simplified form that are further described below in the Detailed Description below. This Summary is not intended to limit the scope of the claimed subject matter nor identify key features or essential features of the claimed subject matter

According to a first aspect, a surgical system is provided comprising: a robotic manipulator; a localizer configured to track the robotic manipulator and an anatomy of a patient; and one or more controllers coupled to the localizer and being configured to: obtain workspace parameters of the robotic manipulator; capture, from the localizer, a current state of the robotic manipulator relative to the anatomy; capture, from the localizer, states of the anatomy in response to movement of the anatomy according to a prescribed manner or a predetermined manner; determine operative parameters of the anatomy based on the captured states of the anatomy; compare the workspace parameters to the operative parameters to determine a desired state for the robotic manipulator relative to the anatomy, whereby in the desired state, the workspace parameters of the robotic manipulator have an acceptable relationship with respect to the operative parameters of the anatomy; and guide placement of the robotic manipulator from the current state to the desired state.

According to a second aspect, a method is provided of operating a surgical system, the surgical system including a robotic manipulator, a localizer configured to track the robotic manipulator and an anatomy of a patient, and one or more controllers coupled to the localizer, and the method comprising the one or more controllers performing the steps of: obtaining workspace parameters of the robotic manipulator; capturing, from the localizer, a current state of the robotic manipulator relative to the anatomy; capturing, from the localizer, states of the anatomy in response to movement of the anatomy according to a prescribed manner or a predetermined manner; determining operative parameters of the anatomy based on the captured states of the anatomy; comparing the workspace parameters to the operative parameters for determining a desired state for the robotic manipulator relative to the anatomy, whereby in the desired state, the workspace parameters of the robotic manipulator have an acceptable relationship with respect to the operative parameters of the anatomy; and guiding placement of the robotic manipulator from the current state to the desired state.

According to a third aspect, a guidance system is provided, comprising: a localizer configured to track a robotic manipulator and an anatomy of a patient; and one or more controllers coupled to the localizer and being configured to: obtain workspace parameters of the robotic manipulator; capture, from the localizer, a current state of the robotic manipulator relative to the anatomy; capture, from the localizer, states of the anatomy in response to movement of the anatomy according to a prescribed manner or a predetermined manner; determine operative parameters of the anatomy based on the captured states of the anatomy; compare the workspace parameters to the operative parameters to determine a desired state for the robotic manipulator relative to the anatomy, whereby in the desired state, the workspace parameters of the robotic manipulator have an acceptable relationship with respect to the operative parameters of the anatomy; and guide placement of the robotic manipulator from the current state to the desired state.

According to a fourth aspect, a non-transitory computer-readable medium is provided that is configured to be utilized with a guidance system comprising a localizer configured to track a robotic manipulator and an anatomy of a patient, wherein the non-transitory computer-readable medium comprises instructions, which when executed by one or more processors, are configured to: obtain workspace parameters of the robotic manipulator; capture, from the localizer, a current state of the robotic manipulator relative to the anatomy; capture, from the localizer, states of the anatomy in response to movement of the anatomy according to a prescribed manner or a predetermined manner; determine operative parameters of the anatomy based on the captured states of the anatomy; compare the workspace parameters to the operative parameters to determine a desired state for the robotic manipulator relative to the anatomy, whereby in the desired state, the workspace parameters of the robotic manipulator have an acceptable relationship with respect to the operative parameters of the anatomy; and guide placement of the robotic manipulator from the current state to the desired state.

According to a fifth aspect, a surgical system is provided, comprising: a robotic manipulator including a plurality of links and joints; a cart supporting the robotic manipulator and comprising a plurality of wheels such that the cart is moveable; and a placement control system coupled to the cart and comprising: a drive system that is configured to drive the wheels; a steering system that is configured to steer the wheels; and one or more controllers configured to control one or both of the drive system and steering system to provide haptic feedback to guide a user in manually moving the cart to a desired location.

According to a sixth aspect, a method of operating the surgical system of the fifth aspect is provided, comprising the one or more controllers controlling one or both of the drive system and steering system for providing haptic feedback to guide a user in manually moving the cart to a desired location.

According to a seventh aspect, a surgical system is provided, comprising: a robotic manipulator including a plurality of links and joints; a cart supporting the robotic manipulator and comprising a plurality of wheels such that the cart is moveable; and a placement control system coupled to the cart and comprising: a drive system that is configured to drive the wheels; a steering system that is configured to steer the wheels; and one or more controllers configured to control the drive system and steering system to autonomously move the cart to a desired location proximate to an anatomy of a patient, and wherein the desired location is determined based on workspace parameters of the robotic manipulator having an acceptable relationship with respect to operative parameters of the anatomy, and wherein the operative parameters of the anatomy are based on movement of the anatomy according to a prescribed manner or a predetermined manner.

According to an eight aspect, a method of operating the surgical system of the seventh aspect is provided, comprising the one or more controllers controlling the drive system and steering system for autonomously moving the cart to a desired location proximate to an anatomy of a patient, and wherein the desired location is determined based on workspace parameters of the robotic manipulator having an acceptable relationship with respect to operative parameters of the anatomy, and wherein the operative parameters of the anatomy are based on movement of the anatomy according to a prescribed manner or a predetermined manner.

According to a ninth aspect, a non-transitory computer-readable medium is provided, comprising instructions, which when executed by one or more processors, are configured to: display, on a display device: a representation of an anatomical joint; graphical instructions to prompt movement of the anatomical joint according to a prescribed manner, wherein the prescribed manner includes one or more of: flexing the anatomical joint; extending the anatomical joint; tilting the anatomical joint; and rotating the anatomical joint; a target indicator comprising one or both of: a target position at which to place the anatomical joint, and a target range within which to place the anatomical joint; a moveable indicator that is configured to move in response to movement of the anatomical joint according to the prescribed manner, and to move relative to one or both of: the target position to provide guidance on relative positioning between the anatomical joint and the target position, and the target range to provide guidance on relative positioning between the anatomical joint and the target range; and a target confirmation indicator that is configured to be displayed in response to the moveable indicator being located at the target position and/or within the target range.

According to a tenth aspect, a guidance system comprising a localizer and the non-transitory computer-readable medium of the ninth aspect is provided. According to an eleventh aspect, a surgical system comprising a robotic manipulator, a localizer and the non-transitory computer-readable medium of the ninth aspect is provided. According to a twelfth aspect, a computer-implemented method is provided of operating any one or more of the non-transitory computer-readable medium of the ninth aspect, the guidance system of the tenth aspect, or the surgical system of the eleventh aspect.

According to a thirteenth aspect, a guidance system is provided for guiding a user in placing a robotic manipulator to a desired state proximate to an anatomy of a patient, the guidance system comprising: a localizer configured to track the robotic manipulator and the anatomy; a display device; and one or more controllers coupled to the localizer and display device and being configured to: obtain workspace parameters of the robotic manipulator; obtain operative parameters of the anatomy; capture, from the localizer, a current state of the robotic manipulator relative to the anatomy; and display, on the display device: a graphical representation of the robotic manipulator that is configured to move in response to changes of the current state of the robotic manipulator captured from the localizer; a graphical representation of the workspace parameters of the robotic manipulator that follow movement of the graphical representation of the robotic manipulator; a graphical representation of the anatomy; a graphical representation of the operative parameters of the anatomy being located proximate to the graphical representation of the anatomy; and a state confirmation indicator that is configured to be displayed in response to determining presence of an acceptable relationship between the graphical representation of the workspace parameters and the graphical representation of the operative parameters.

According to a fourteenth aspect, a surgical system comprising the robotic manipulator and the guidance system of the thirteen aspect is provided. According to a fifteenth aspect, a method of operating the guidance system of the thirteenth aspect is provided. According to a sixteen aspect, a method of operating the surgical system of the fourteen aspect is provided.

Any of the above aspects can be combined in part or in whole with any other aspect.

Any of the above aspects, whether combined in part or in whole, can be further combined with any of the following implementations, in full or in part.

In one implementation, the localizer tracks the anatomy by tracking states of at least one bone, and optionally, two bones forming a portion of an anatomical joint. In one implementation, the localizer tracks external portions of the anatomy. In one implementation, the localizer is configured to track the states of the robotic manipulator and/or the anatomy using any one or more of: an infrared tracking system; a machine vision system; a radio frequency tracking system; an ultrasound tracking system; and an electromagnetic tracking system. In one implementation, the one or more controllers capture, from the localizer, the states of one or two bones in response to movement thereof. In one implementation, the one or more controllers determine operative parameters of the anatomical joint based on the captured states of the one or two bones of the joint. In one implementation, the one or more controllers prompt a user, on a display device, to manually move the anatomy. In one implementation, the one or more controllers capture, from the localizer, the states of the anatomy in response to manual movement of the anatomy. In one implementation, the one or more controllers prompt the user, on the display device, to manually move the anatomy in a prescribed/recommended manner. In one implementation, the one or more controllers capture, from the localizer, the states of the anatomy in response to movement of the anatomy in the prescribed/recommended manner.

In one implementation, the anatomy comprises an anatomical joint, including but not limited to, a knee joint, a hip joint, a shoulder joint, an ankle joint, an elbow joint, or a spinal joint, and the prescribed/recommended manner includes one or more of: flexing and extending the anatomical joint; tilting the anatomical joint; and rotating the anatomical joint. In one implementation, the one or more controllers determine the operative parameters of the anatomical joint based on any one or more of: the states of the anatomical joint being captured at physical range of motion limits of the anatomical joint; states of the anatomical joint being captured during continuous motion of the anatomical joint; states of the anatomical joint being captured at one or more discrete positions within physical range of motion limits of the anatomical joint; and based on augmentation of the captured states with statistical data. In one implementation, the one or more controllers determine the operative parameters of the anatomy based on any one or more of the following: patient data; surgical plan data; and statistical data.

In one implementation, an anatomical manipulator is configured to support and move the anatomical joint, and optionally, in the predetermined manner. In one implementation, the one or more controllers are coupled to the anatomical manipulator and command the anatomical manipulator to autonomously or semi-autonomously move the anatomical joint. In one implementation, the one or more controllers capture the states of the anatomical manipulator in response to, or during, autonomous or semi-autonomous movement of the anatomical joint by the anatomical manipulator. In one implementation, the one or more controllers predict the operative parameters of the anatomy and predictions can be performed using captured states as an input or without using any prior captured states of the anatomy.

In one implementation, the robotic manipulator comprises a robotic arm including a plurality of links and joints. In one implementation, the robotic manipulator comprises a cart that supports the robotic arm. In one implementation, the cart comprises a plurality of wheels such that the cart is moveable. In one implementation, the robotic manipulator is table mounted or patient mounted. In one implementation, the robotic manipulator is mounted to a passive, articulated, holding arm. In one implementation, the robotic manipulator is hand-held and supported by a user against the force of gravity, and optionally selectively connected to an adjustable arm. In one implementation, the robotic manipulator is moveably coupled to a surgical boom. In one implementation, the robotic manipulator is coupled to an imaging device or gantry that is moveable.

In one implementation, the robotic manipulator is configured to be manually moved by user. In one implementation, the one or more controllers guide placement of the robotic manipulator by displaying, on a display device, instructions to assist a user to manually move the robotic manipulator from the current state to the desired state. In one implementation, the one or more controllers guide placement of the cart of the robotic manipulator from the current state to the desired state. In one implementation, the desired state is a location of the manipulator, base of the manipulator, or cart that supports the manipulator. In one implementation, the desired state is a pose of the manipulator arm. In one implementation, the desired state is both a location of the manipulator and a pose of the manipulator arm. In one implementation, the one or more controllers are configured to communicate with the manipulator controller and to guide placement of the robotic arm by being configured to instruct the manipulator controller to autonomously move the robotic arm from the current state to the desired state. In one implementation, the one or more controllers compare the workspace parameters to the operative parameters to determine the desired state for the robotic manipulator, whereby in the desired state, the workspace parameters of the robotic manipulator have an acceptable relationship to the operative parameters of the anatomy, and optionally, fully encompass the operative parameters of the anatomy or at least encompass the operative parameters of the anatomy within a predefined threshold of acceptability. In one implementation, the one or more controllers obtain the workspace parameters of the robotic manipulator by obtaining one or more of: a predetermined kinematic model of the robotic manipulator; factory data related to the robotic manipulator; calibration or setup data related to the robotic manipulator; and surgical plan data related to the robotic manipulator.

In one implementation, the one or more controllers display, on a display device a representation of the anatomy, and optionally, graphical instructions to prompt movement of the anatomy, and optionally, a representation of movement of the anatomy. In one implementation, the one or more controllers display, on the display device, a target indicator. In one implementation, the target indicator is a target position at which to place the anatomy. In one implementation, the target indicator is a target range within which to place the anatomy. In one implementation, the one or more controllers display, on the display device, a moveable indicator that is configured to move in response to movement of the anatomy and to move relative to one or both of: the target position to provide visual guidance on relative positioning between the anatomy and the target position; and the target range to provide visual guidance on relative positioning between the anatomy and the target range. In one implementation, the one or more controllers display, on the display device, a target confirmation indicator that is configured to be displayed in response to the moveable indicator being located at the target position and/or within the target range. In one implementation, the target indicator is a scrolling bar that shows a desired range of motion to be captured for the anatomical joint. In one implementation, the moveable indicator is configured to move along the scrolling bar. In one implementation, the target confirmation indicator is implemented by changing a color of the target indicator. In one implementation, one or more controllers instruct the display device to display graphical instructions to subsequently prompt movement of the anatomical joint according to a second prescribed manner, different from the first prescribed manner, in response to the target confirmation indicator successfully being displayed in response to movement of the anatomical joint according to the first prescribed manner. In one implementation, in response to the target confirmation indicator failing to be displayed in response to movement of the anatomical joint according to the first prescribed manner, the one or more controllers re-prompt movement of the anatomical joint according to the first prescribed manner, and/or prevent subsequent prompt of movement of the anatomical joint according to the second prescribed manner, and optionally continue to prevent so until the target confirmation indicator is successfully displayed in response to movement of the anatomical joint according to the first prescribed manner.

In one implementation, the one or more controllers guide placement of the robotic manipulator from the current state to the desired state by being configured to display, on a display device: a representation of the robotic manipulator at the current state; a representation of the anatomy; a graphical representation of the desired state of the robotic manipulator; movement of the representation of the robotic manipulator from the current state to the desired state. In one implementation, the controller(s) display a state confirmation indicator that is configured to be displayed in response to the representation of the robotic manipulator reaching the desired state. In one implementation, any displayed representation can be actual or graphical. In one implementation, any displayed representation can be 2D or 3D and from any perspective. In one implementation, the one or more controllers guide placement of the robotic manipulator from the current state to the desired state by displaying, on a display device a graphical representation of the workspace parameters of the robotic manipulator. In one implementation, the one or more controllers display a graphical representation of the operative parameters of the anatomy. In one implementation, the one or more controllers display a state confirmation indicator that is configured to be displayed in response to determining presence of an acceptable relationship between the graphical representation of the workspace parameters and the graphical representation of the operative parameters. In one implementation, the anatomy is subject to a surgical procedure involving a plurality of steps, and wherein, after successful placement of the robotic manipulator from the current state to the desired state, the one or more controllers identify a change to one or both of: the workspace parameters and the operative parameters. In one implementation, the controller(s) evaluate the change to determine a second desired state for the robotic manipulator and guide placement of the robotic manipulator from the current state to the second desired state. In one implementation, the controller(s) evaluate the change to determine a desired pose of the anatomy and guide placement of the anatomy to the desired pose.

In one implementation, a sensing system can be coupled to the manipulator or cart or proximate to the cart to detect an environment around the manipulator or cart. In one implementation, a sensing system can be located near the anatomy to detect the anatomy and an environment of the anatomy. In one implementation, the sensing system is configured to detect a current state of the cart. In one implementation, a placement control system is located on the cart and comprises a drive system that is configured to drive the wheels. In one implementation, the placement control system includes a steering system that is configured to steer the wheels. In one implementation, the placement control system includes a cart controller that is configured to control the drive system and steering system. In one implementation, the placement control system and/or the one or more controllers are configured to guide placement of the robotic manipulator to the desired state. In one implementation, the placement control system controls the drive system and/or steering system to autonomously move the cart from the current state to the desired state. In one implementation, the placement control system provides haptic feedback to guide a user on placement of the robotic manipulator from the current state to the desired state. In one implementation, the one or more controllers are configured to virtually define a haptic path from a current location of the cart to the desired location; and control the one or both of the drive system and steering system to provide haptic feedback in response the user manually moving the cart in a manner that interacts with, or deviates from, the haptic path. In one implementation, the one or more controllers are configured to: virtually define a haptic zone proximate to the desired location; and control the one or both of the drive system and steering system to provide haptic feedback in response the user manually moving the cart in a manner that interacts with, or deviates from, the haptic zone. In one implementation, in response the user manually moving the cart in a manner that interacts with, or deviates from, one or both of the haptic path or the haptic zone, the one or more controllers control the one or both of the drive system and steering system to provide haptic feedback by being configured to perform any one or more of the following: control the steering system and/or drive system to restrict turning of the wheels; control the steering system to offset the deviation; control the steering system and/or drive system to vibrate the wheels; and control the steering system to vibrate the steering controls. In one implementation, the placement control system is coupled to the sensing system and is configured to: detect, from the sensing system, the current location of the cart; generate directions to move the cart from the current location to the desired location; and generate the haptic path and/or haptic zone based on the generated directions. In one implementation, the haptic path and/or haptic zone are graphically displayed on a display device.

In one implementation audible, haptic, and/or visual feedback can be provided to the user to guide placement. In one implementation, the display device of any aspect is a head mounted device that is configured to graphically display, using mixed reality or augmented reality, the desired state of the robotic manipulator, the operative parameters, the workspace parameters, or any of the displayed information above.

Any of the implementations described above can be combined in part or in whole and can be utilized with any aspect.

### BRIEF DESCRIPTION OF THE DRAWINGS

Advantages of the present invention will be readily appreciated as the same becomes better understood by reference to the following detailed description when considered in connection with the accompanying drawings.

FIG. 1 is a perspective view of one implementation of a surgical system shown relative to a patient, wherein the surgical system comprises a robotic manipulator on a cart and a navigation system including a localizer.

FIG. 2 is a block diagram of a control system for controlling the surgical system, according to one implementation.

FIG. 3 is a functional block diagram of a software program for controlling the surgical system, according to one implementation.

FIG. 4 is a perspective view of one implementation of the manipulator comprising a placement control system to aide in guiding the manipulator to a desired state.

FIG. 5 is a perspective view of a patient on a surgical table whereby operative parameters of the patient anatomy are graphically illustrated, according to one implementation.

FIG. 6 is a block diagram of one implementation of a method of guiding placement of the manipulator from a current state to a desired state.

FIG. 7 is block diagram that continues the method of guiding placement of the manipulator from a current state to a desired state, according to one implementation.

FIG. 8 is a perspective view of a kinematic chain of the manipulator whereby workspace parameters of the manipulator are graphically illustrated, according to one implementation.

FIG. 9 is a display screen showing a GUI that aides in guiding a user to move the anatomy (e.g., flexing a knee) using target and moving indicators, according to one implementation.

FIG. 10 is a display screen showing a GUI that aides in guiding a user to move the anatomy (e.g., tilting a knee) using target and moving indicators, according to one implementation.

FIG. 11 is a side view of one implementation of an anatomical manipulator that is configured to support or automatically move the anatomy (e.g., extending a knee).

FIG. 12 is a front view of the anatomical manipulator of FIG. 11 that is configured to support or automatically move the anatomy (e.g., tilting a knee).

FIG. 13 is a display screen showing a GUI that aides in guiding placement of the manipulator and/or cart to a desired state, according to one implementation.

FIG. 14 is a display screen showing a GUI that aides in guiding placement of the manipulator and/or cart to a desired state, according to another implementation.

### DETAILED DESCRIPTION

### I. Example System Overview

Referring to the Figures, wherein like numerals indicate like or corresponding parts throughout the several views, a surgical robotic system (hereinafter “system”) 10 and method for operating the same are shown throughout.

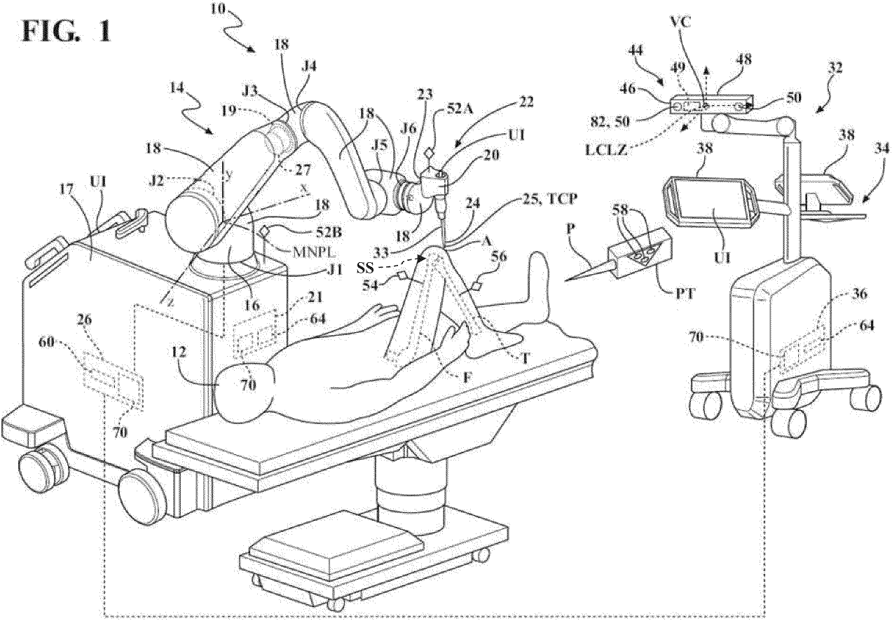

Referring to FIG. 1, an example of the surgical robotic system 10 is illustrated. The system 10 is useful for treating a surgical site SS or anatomical volume (A) of a patient 12, such as treating bone or soft tissue. In FIG. 1, the patient 12 is undergoing a surgical procedure. The anatomy in FIG. 1 includes a femur F and a tibia T of the patient 12. The surgical procedure may involve tissue removal or other forms of treatment. Treatment may include cutting, coagulating, lesioning the tissue, other in-situ tissue treatments, or the like. In some examples, the surgical procedure involves partial or total knee or hip replacement surgery, shoulder replacement surgery, spine surgery, or ankle surgery. In some examples, the system 10 is designed to cut away material to be replaced by surgical implants, such as hip and knee implants, including unicompartmental, bicompartmental, multicompartmental, or total knee implants. Some of these types of implants are shown in U.S. Pat. Application Publication No. 2012/0330429, entitled, “Prosthetic Implant and Method of Implantation,” the disclosure of which is hereby incorporated by reference. The system 10 and techniques disclosed herein may be used to perform other procedures, surgical or non-surgical. The system 10 may be used in industrial (non-surgical) applications or other applications where robotic systems are utilized.

In the implementation shown, the system 10 includes a (robotic) manipulator 14. The manipulator 14 has a base 16 and plurality of links 18. A cart 17 supports the manipulator 14 such that the manipulator 14 is supported by the cart 17. The links 18 collectively form one or more arms of the manipulator 14. In some implementations, one or more of the links 18 is a trackable link that includes tracking elements such as LEDs. The manipulator 14 may have a serial arm configuration (as shown in FIG. 1), a parallel arm configuration, or any other suitable manipulator configuration. In other examples, more than one manipulator 14 may be utilized in a multiple arm configuration.

In the example shown in FIG. 1, the manipulator 14 comprises a plurality of joints J and a plurality of joint encoders 19 located at the joints J for determining position data of the joints J. For simplicity, only one joint encoder 19 is illustrated in FIG. 1, although other joint encoders 19 may be similarly illustrated. The manipulator 14 according to one example has six joints J1-J6 implementing at least six-degrees of freedom (DOF) for the manipulator 14. However, the manipulator 14 may have any number of degrees of freedom and may have any suitable number of joints J and may have redundant joints. In one example, the manipulator 14 can have a configuration such as the robotic manipulator described in US Pat. No. 10,327,849, entitled “Robotic System and Method for Backdriving the Same”, the contents of which are hereby incorporated by reference in its entirety.

The manipulator 14 need not require joint encoders 19 but may alternatively, or additionally, utilize motor encoders present on motors 27 at each joint J. Also, the manipulator 14 need not require rotary joints, but may alternatively, or additionally, utilize one or more prismatic joints. Any combination of joint types is contemplated.

The base 16 of the manipulator 14 is a portion of the manipulator 14 that provides a fixed reference coordinate system for other components of the manipulator 14 or the system 10 in general. The origin of a manipulator coordinate system MNPL may be defined at the fixed reference of the base 16. The base 16 may be defined with respect to any suitable portion of the manipulator 14, such as one or more of the links 18. Alternatively, or additionally, the base 16 may be defined with respect to the cart 17, such as where the manipulator 14 is physically attached to the cart 17. In one example, the base 16 is defined at an intersection of the axes of joints J1 and J2. Thus, although joints J1 and J2 are moving components in reality, the intersection of the axes of joints J1 and J2 is nevertheless a virtual fixed reference pose, which provides both a fixed position and orientation reference and which does not move relative to the manipulator 14 and/or cart 17.

In other examples, the manipulator 14 can be a hand-held manipulator where the base 16 is a base portion of a tool (e.g., a portion held free-hand by a user against the force of gravity) and the tool tip is movable relative to the base portion. The base portion has a reference coordinate system that is tracked and the tool tip has a tool tip coordinate system that is computed relative to the reference coordinate system (e.g., via motor and/or joint encoders and forward kinematic calculations). Movement of the tool tip can be controlled to follow the path since its pose relative to the path can be determined. The hand-held manipulator 14 can be attachable to and supported by an adjustable arm. The adjustable arm can be motorized or passive and manually lockable.

In another example, the manipulator 14 can be mounted to an imaging device or gantry, such as a CT, X-Ray, or Fluoroscopy imaging device or scanner. One example of a manipulator 14 that can be utilized with an imaging device can be like that described in U.S. Pat. No. 11,103,990, entitled “System and Method for Mounting a Robotic Arm in a Surgical Robotic System” the contents of which are hereby incorporated by reference in its entirety. In yet another example, the manipulator 14 can be mounted to a ceiling or moveable overhead unit, such as a surgical boom. The manipulator 14 can be coupled to any other object not specifically described herein.

The manipulator 14 and/or cart 17 house a manipulator controller 26, or other type of control unit. The manipulator controller 26 may comprise one or more computers, or any other suitable form of controller that directs the motion of the manipulator 14. The manipulator controller 26 may have a central processing unit (CPU) and/or other processors, memory, and storage. The manipulator controller 26 is loaded with software as described below. The processors could include one or more processors to control operation of the manipulator 14. The processors can be any type of microprocessor, multi-processor, and/or multi-core processing system. The manipulator controller 26 may additionally, or alternatively, comprise one or more microcontrollers, field programmable gate arrays, systems on a chip, discrete circuitry, and/or other suitable hardware, software, or firmware that is capable of carrying out the functions described herein. The term processor is not intended to limit any implementation to a single processor. The manipulator 14 may also comprise a user interface UI with one or more displays and/or input devices (e.g., push buttons, keyboard, mouse, microphone (voice-activation), gesture control devices, touchscreens, etc.).

A tool 20 can couple to the manipulator 14 and is movable relative to the base 16 to interact with the anatomy in certain modes. The tool 20 is a surgical tool and is or forms part of an end effector 22 supported by the manipulator 14 in certain implementations. The end effector 22 can also be a tool holder such as a slotted cut saw cut guide, a guide tube, an impactor support, or any other type of holder that removably receives the tool 20, or the like. The manipulator 14 may include a first mounting interface configured to removably receive the end effector 22. In order to secure to the first mounting interface, the end effector 22 may include end effector body 23 which includes a second mounting interface configured to couple to the first mounting interface. The tool 20 may be grasped by the user. One possible arrangement of the manipulator 14 and the tool 20 is described in U.S. Pat. No. 9,119,655, entitled, “Surgical Manipulator Capable of Controlling a Surgical Instrument in Multiple Modes,” the disclosure of which is hereby incorporated by reference. The manipulator 14 and the tool 20 may be arranged in alternative configurations. The tool 20 can be like that shown in U.S. Pat. No. 9,566,121, filed on Mar. 15, 2014, entitled, “End Effector of a Surgical Robotic Manipulator,” hereby incorporated by reference.

The tool 20 may include an energy applicator 24 designed to contact and remove the tissue of the patient 12. In one example, the energy applicator 24 is a bur 25. The bur 25 may be substantially spherical and may have a spherical center, radius (r) and diameter. Alternatively, the energy applicator 24 may be a drill bit, a saw blade, an impactor, a reamer, an ultrasonic vibrating tip, or the like. The tool 20 and/or energy applicator 24 may comprise any geometric feature, e.g., perimeter, circumference, radius, diameter, width, length, volume, area, surface/plane, range of motion envelope (along any one or more axes), etc. The geometric feature may be considered to determine how to locate the tool 20 relative to the tissue at the surgical site SS to perform the desired treatment. In some of the implementations described herein, a spherical bur having a tool center point (TCP) will be described for convenience and ease of illustration but is not intended to limit the tool 20 to any particular form.

The tool 20 may comprise a tool controller 21 to control operation of the tool 20, such as to control power to the tool (e.g., to a rotary motor of the tool 20), control movement of the tool 20, control irrigation/aspiration of the tool 20, and/or the like. The tool controller 21 may be in communication with the manipulator controller 26 or other components. The tool 20 may also comprise a user interface UI with one or more displays and/or input devices (e.g., push buttons, keyboard, mouse, microphone (voice-activation), gesture control devices, touchscreens, etc.). The manipulator controller 26 controls a state (position and/or orientation) of the tool 20 (e.g., the TCP) with respect to a coordinate system, such as the manipulator coordinate system MNPL. The manipulator controller 26 can control (linear or angular) velocity, acceleration, or other derivatives of motion of the tool 20.

The tool center point (TCP), in one example, is a predetermined reference point defined at the energy applicator 24. The TCP has a known, or able to be calculated (i.e., not necessarily static), pose relative to other coordinate systems. The geometry of the energy applicator 24 is known in or defined relative to a TCP coordinate system. The TCP may be located at the spherical center of the bur 25 of the tool 20 such that only one point is tracked. The TCP may be defined in several ways depending on the configuration of the energy applicator 24. The manipulator 14 could employ the joint/motor encoders, or any other non-encoder position sensing method, to enable a pose of the TCP to be determined. The manipulator 14 may use joint measurements to determine TCP pose and/or could employ techniques to measure TCP pose directly. The control of the tool 20 is not limited to a center point. For example, any suitable primitives, meshes, etc., can be used to represent the tool 20.

The system 10 further includes a navigation system 32. One example of the navigation system 32 is described in U.S. Pat. No. 9,008,757, entitled, “Navigation System Including Optical and Non-Optical Sensors,” hereby incorporated by reference. The navigation system 32 tracks movement of various objects. Such objects include, for example, the manipulator 14, the tool 20 and the anatomy, e.g., femur F and tibia T. The navigation system 32 tracks these objects to gather state information of each object with respect to a (navigation) localizer coordinate system LCLZ. Coordinates in the localizer coordinate system LCLZ may be transformed to the manipulator coordinate system MNPL, and/or vice-versa, using transformations.

The navigation system 32 may include a cart assembly 34 that houses a navigation controller 36, and/or other types of control units. A navigation user interface UI is in operative communication with the navigation controller 36. The navigation user interface includes one or more displays 38. The navigation system 32 is capable of displaying a graphical representation of the relative states of the tracked objects to the user using the one or more displays 38. The navigation user interface UI further comprises one or more input devices to input information into the navigation controller 36 or otherwise to select/control certain aspects of the navigation controller 36. Such input devices include interactive touchscreen displays. However, the input devices may include any one or more of push buttons, a keyboard, a mouse, a microphone (voice-activation), gesture control devices, and the like.

The navigation system 32 also includes a localizer 44 coupled to the navigation controller 36. The relative location of the localizer 44 with respect to the manipulator 14 in FIG. 1 is provided only for illustrative purposes to show the respective components and is not necessarily representative of the optimal manner in which to setup the localizer 44. In one example, the localizer 44 is an optical localizer and includes a camera unit 46. The camera unit 46 has an outer casing 48 that houses one or more sensors 50, such as optical sensors. The localizer 44 may comprise its own localizer controller 49 and may further comprise a video camera VC. The localization device 44 may include an IR transmitter 82 configured to send and receive infrared (IR) signals. The IR transmitter 82 is in communication with the localizer controller 49 such that signals received by the IR transmitter 82 can be relayed to the localizer controller 49. As described in more detail below, the IR transmitter 82 may be in communication with the various trackers utilized by the surgical robotic system 10. Any IR communications from the localizer 44 may originate from the IR transmitter 82.

The navigation system 32 includes one or more trackers. In one example, the trackers include a pointer tracker PT, one or more robotic or tool trackers 52A, 52B, a first patient tracker 54, and a second patient tracker 56. The trackers may include one or more trackable elements arranged in a unique tracking geometry such that the localizer 44 can differentiate the trackers from one another. Any one or more of the trackers 52A, 52B, 54, 56, PT may include active markers 58. The active markers 58 may include light emitting diodes (LEDs). The LEDs may be configured to provide tracking information to the navigation system 32, and the photosensors may be configured to receive signals from the navigation system 32. Alternatively, the trackers 52A, 52B, 54, 56, PT may have passive markers, such as reflectors, which reflect light emitted from the camera unit 46. Other suitable markers not specifically described herein may be utilized. Any one or more of the trackers 52A, 52B, 54, 56, PT may include photosensors or infrared receivers to receive control signals from the navigation system 32.

In the implementation shown, the first patient tracker 54 is firmly affixed to the femur F of the patient 12, and the second patient tracker 56 is firmly affixed to the tibia T of the patient 12. In this example, the patient trackers 54, 56 are firmly affixed to sections of bone. However, there may be methods to track the patient 12 anatomy without firmly affixing trackers to bone. For instance, ultrasound tracking devices may surround the skin of the limbs to non-invasively track the limbs. The pointer tracker PT is firmly affixed to a pointer P used for registering the anatomy to the localizer coordinate system LCLZ.

The tracker 52A, herein referred to as an end effector tracker 52A, may be secured to any part of the end effector 22. For example, the end effector tracker 52A may be secured to the end effector body 23 or the tool 20. In addition, the end effector tracker 52A may be integrated into the end effector 22 or one of the mounting interfaces. The end effector tracker 52A may comprise one light emitting diode or a plurality of light emitting diodes integrated into or coupled to the end effector body 23.

The tracker 52B, herein referred to as a base tracker 52B, may be moveable relative to the base 16 and may be placed in a stowed position relative to the base 16. For example, the base 16 may further include an adjustable arm configured to support the base tracker 52B. The adjustable arm may include a tracker interface configured to couple to the base tracker 52B. The adjustable arm may be pivotably secured to the base 16 at a connection point such that the adjustable arm may be moved between a stowed position and various deployed positions. The adjustable arm may be considered to be in the stowed position when it is folded flat up against the base, and the adjustable arm may be considered to be in one of the deployed positions when it is pivoted about the connection point so as to form an angle with the side of the base 16. Such as arrangement allows the base tracker 52B to be coupled to the adjustable arm at the tracker interface and moved relative to the base 16 until the tracker 52B is in a desired position. In an alternative configuration, the base tracker 52B is located on one or more of the links 18 of the manipulator 14.

The localizer 44 tracks the trackers 52A, 52B, 54, 56, PT to determine a state of each of the trackers 52A, 52B, 54, 56, PT, which correspond respectively to the state of the object respectively attached thereto. The localizer 44 may perform triangulation techniques to determine the states of the trackers 52A, 52B, 54, 56, PT, and associated objects. The localizer 44 provides the state of the trackers 52A, 52B, 52C, 54, 56, PT to the navigation controller 36. In one example, the navigation controller 36 determines and communicates the state the trackers 52A, 52B, 54, 56, PT to the manipulator controller 26. As used herein, the state of an object includes, but is not limited to, data that defines the position and/or orientation of the tracked object or equivalents/derivatives of the position and/or orientation. For example, the state may be a pose of the object, and may include linear velocity data, and/or angular velocity data, and the like.

The navigation controller 36 may comprise one or more computers, or any other suitable form of controller. Navigation controller 36 has a central processing unit (CPU) and/or other processors, memory (not shown), and storage (not shown). The processors can be any type of processor, microprocessor, or multi-processor system. The navigation controller 36 is loaded with software. The software, for example, converts the signals received from the localizer 44 into data representative of the position and orientation of the objects being tracked. The navigation controller 36 may additionally, or alternatively, comprise one or more microcontrollers, field programmable gate arrays, systems on a chip, discrete circuitry, and/or other suitable hardware, software, or firmware that is capable of carrying out the functions described herein.

Although one example of the navigation system 32 is shown that employs triangulation techniques to determine object states, the navigation system 32 may have any other suitable configuration for tracking the manipulator 14, tool 20, and/or the patient 12.

In another example, the navigation system 32 and/or localizer 44 are radio frequency (RF)-based. For example, the navigation system 32 may comprise an RF transceiver coupled to the navigation controller 36. The manipulator 14, the tool 20, and/or the patient 12 may comprise RF emitters or transponders attached thereto. The RF emitters or transponders may be passive or actively energized. The RF transceiver transmits an RF tracking signal and generates state signals to the navigation controller 36 based on RF signals received from the RF emitters. The navigation controller 36 may analyze the received RF signals to associate relative states thereto. The RF signals may be of any suitable frequency. The RF transceiver may be positioned at any suitable location to track the objects using RF signals effectively. Furthermore, the RF emitters or transponders may have any suitable structural configuration that may be much different than the trackers 52A, 52B, 52C, 54, 56, PT shown in FIG. 1.

In another example, the navigation system 32 and/or localizer 44 are electromagnetically based. For example, the navigation system 32 may comprise an EM transceiver coupled to the navigation controller 36. The manipulator 14, the tool 20, and/or the patient 12 may comprise EM components attached thereto, such as any suitable magnetic tracker, electro-magnetic tracker, inductive tracker, or the like. The trackers may be passive or actively energized. The EM transceiver generates an EM field and generates state signals to the navigation controller 36 based upon EM signals received from the trackers. The navigation controller 36 may analyze the received EM signals to associate relative states thereto. Again, such navigation system 32 examples may have structural configurations that are different than the navigation system 32 configuration shown in FIG. 1.

In yet another example, the navigation system 32 and/or localizer 44 are machine vision or computer vision based. For example, the navigation system 32 may comprise a machine or computer vision camera coupled to the navigation controller 36. The manipulator 14, the tool 20, and/or the patient 12 may comprise vision detectable elements attached thereto, such as any suitable pattern, color, barcode, QR code, or the like. The vision detectable elements may be passive or actively energized. The navigation controller 36 may analyze image and/or depth data from the vision detectable elements to associate relative states thereto. Again, such navigation system 32 examples may have structural configurations that are different than the navigation system 32 configuration shown in FIG. 1.

In yet another example, the navigation system 32 and/or localizer 44 are ultrasound based. For example, the navigation system 32 may comprise an ultrasound tracker coupled to the navigation controller 36. The manipulator 14, the tool 20, and/or the patient 12 may be detectable the ultrasound tracking. The navigation controller 36 may analyze ultrasound image data to associate relative states thereto.

The navigation system 32 can use any combination of the above-described localization techniques for hybrid modality tracking. The navigation system 32 may have any other suitable components or structure not specifically recited herein. Furthermore, any of the techniques, methods, and/or components described above with respect to the navigation system 32 shown may be implemented or provided for any of the other examples of the navigation system 32 described herein.

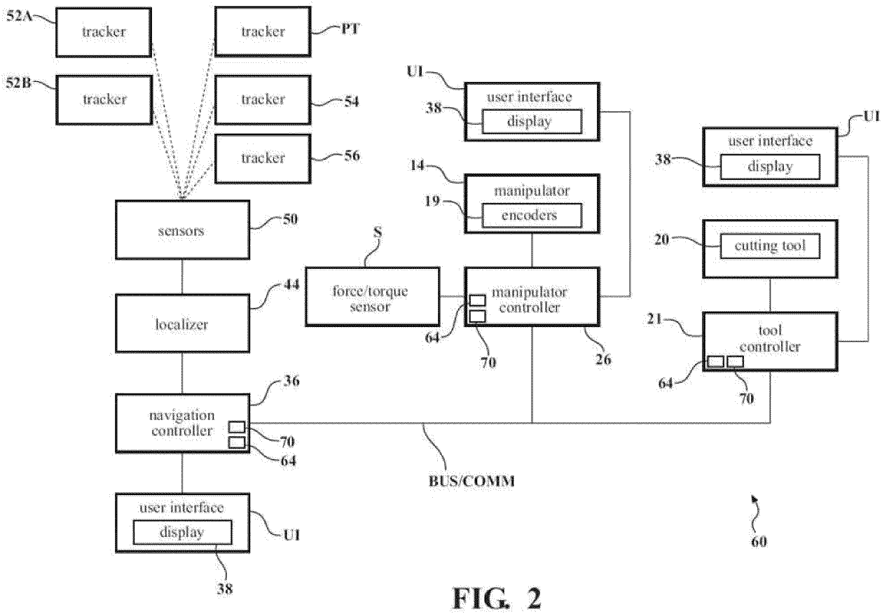

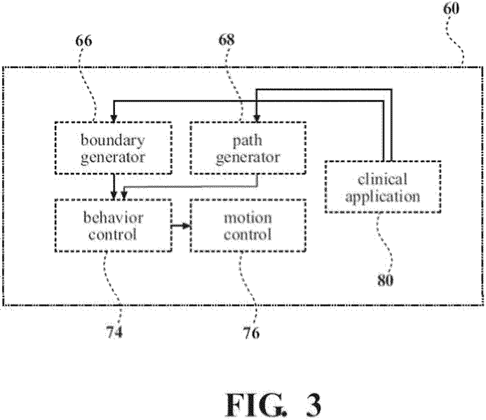

Referring to FIG. 2, one implementation of the system 10 may include a control system 60 that comprises, among other components, the manipulator controller 26, the navigation controller 36, and the tool controller 21. The control system 60 may further include one or more software programs and software modules shown in FIG. 3. The software modules may be part of the program or programs that operate on the manipulator controller 26, navigation controller 36, tool controller 21, or any combination thereof, to process data to assist with control of the system 10. The software programs and/or modules include computer-readable instructions stored in non-transitory memory 64 on the manipulator controller 26, navigation controller 36, tool controller 21, or a combination thereof, to be executed by one or more processors 70 of the controllers 21, 26, 36. The memory 64 may be any suitable configuration of memory, such as RAM, non-volatile memory, etc., and may be implemented locally or from a remote database. Additionally, software modules for prompting and/or communicating with the user may form part of the program or programs and may include instructions stored in memory 64 on the manipulator controller 26, navigation controller 36, tool controller 21, or any combination thereof. The user may interact with any of the input devices of the navigation user interface UI or other user interface UI to communicate with the software modules. The user interface software may run on a separate device from the manipulator controller 26, navigation controller 36, and/or tool controller 21.

The control system 60 may comprise any suitable configuration of input, output, and processing devices suitable for carrying out the functions and methods described herein. The control system 60 may comprise the manipulator controller 26, the navigation controller 36, or the tool controller 21, or any combination thereof, or may comprise only one of these controllers. These controllers may communicate via a wired bus or communication network as shown in FIG. 2, via wireless communication, or otherwise. The control system 60 may also be referred to as a controller. The control system 60 may comprise one or more microcontrollers, field programmable gate arrays, systems on a chip, discrete circuitry, sensors, displays, user interfaces, indicators, and/or other suitable hardware, software, or firmware that is capable of carrying out the functions described herein.

Referring to FIG. 3, the software employed by the control system 60 may include a boundary generator 66 and a path generator 68. The boundary generator 66 is a software program or module that generates a virtual boundary for constraining movement and/or operation of the tool 20. The manipulator controller 26 and/or the navigation controller 36 tracks and/or controls/positions the state of the tool 20 relative to the virtual boundaries. To that end, the path generator 68 may generate a milling/tool path for the tool 20 to traverse, such as for removing sections of the anatomy to receive an implant. In one example, the tool path is defined as a tissue removal path, but, in other versions, the tool path may be used for treatment other than tissue removal. For instance, the tool path may be utilized to setup or configure the manipulator 14 to a specified position or starting location. The boundary generator 66 and the path generator 68 may each be implemented on the manipulator controller 26. Alternatively, the boundary generator 66 and/or the path generator 68 may be implemented on other components, such as the navigation controller 36. One example of a system and method for generating the virtual boundaries and/or the tool path is described in U.S. Pat. No. 9,119,655, entitled, “Surgical Manipulator Capable of Controlling a Surgical Instrument in Multiple Modes.” Another example of such a system/method is described in U.S. Pat. Publication No. 2020/0281676, entitled, “Systems and Methods for Controlling Movement of a Surgical Tool Along a Predefined Path.” The disclosures of both of which are hereby incorporated by reference.

Two additional software programs or modules may be run on the manipulator controller 26 and/or the navigation controller 36. One software module performs behavior control 74. Behavior control 74 is the process of computing data that indicates the next commanded position and/or orientation (e.g., pose) for the tool 20. Output from the boundary generator 66, the path generator 68, and a force/torque sensor S (coupled between the end effector and the manipulator) may feed as inputs into the behavior control 74 to determine the next commanded position and/or orientation for the tool 20. The behavior control 74 may process these inputs, along with one or more virtual constraints described further below, to determine the commanded pose. The second software module performs motion control 76. One aspect of motion control is the control of the manipulator 14. The motion control 76 receives data defining the next commanded pose from the behavior control 74. Based on these data, the motion control 76 determines the next position of the joint angles of the joints J of the manipulator 14 (e.g., via inverse kinematics and Jacobian calculators) so that the manipulator 14 is able to position the tool 20 as commanded by the behavior control 74, e.g., at the commanded pose. One example of such software modules is described in U.S. Pat. Publication No. 2020/0281676, incorporated above.

Additionally, the user interface UI can be a clinical application 80 provided to handle user interaction. The clinical application 80 handles many aspects of user interaction and coordinates the surgical workflow, including pre-operative planning, manipulator setup, tracker setup, localizer setup, implant placement, registration, bone preparation visualization, post-operative evaluation of implant fit, and navigation settings, control, calibration, validation, etc. The clinical application 80 is configured to be output to the displays 38. The clinical application 80 may run on its own separate processor or may run alongside the navigation controller 36. An example of the clinical application 80 is described in U.S. Pat. Publication No. 2020/0281676, incorporated above.

The system 10 may operate in a manual mode, such as described in U.S. Pat. No. 9,119,655, incorporated above. Here, the user manually directs, and the manipulator 14 executes movement of the tool 20 and its energy applicator 24 at the surgical site SS. The user physically contacts the tool 20 to cause movement of the tool 20 in the manual mode. In one version, the manipulator 14 monitors forces and torques placed on the tool 20 by the user in order to position the tool 20. For example, the manipulator 14 may comprise the force/torque sensor S that detects the forces and torques applied by the user and generates corresponding input used by the control system 60 (e.g., one or more corresponding input/output signals).

The force/torque sensor S may comprise a 6-DOF force/torque transducer. The manipulator controller 26 and/or the navigation controller 36 receives the input (e.g., signals) from the force/torque sensor S. In response to the user-applied forces and torques, the manipulator 14 moves the tool 20 in a manner that emulates the movement that would have occurred based on the forces and torques applied by the user. Movement of the tool 20 in the manual mode may also be constrained in relation to the virtual boundaries generated by the boundary generator 66. In some versions, measurements taken by the force/torque sensor S are transformed from a force/torque coordinate system FT of the force/torque sensor S to another coordinate system, such as a virtual mass coordinate system in which a virtual simulation is carried out on the virtual rigid body model of the tool 20 so that the forces and torques can be virtually applied to the virtual rigid body in the virtual simulation to ultimately determine how those forces and torques (among other inputs) would affect movement of the virtual rigid body, as described below.

The system 10 may also operate in a semi-autonomous mode in which the manipulator 14 moves the tool 20 along the milling path 72 (e.g., the active joints J of the manipulator 14 operate to move the tool 20 without requiring force/torque on the tool 20 from the user). An example of operation in the semi-autonomous mode is also described in U.S. Pat. No. 9,119,655, incorporated above. In some implementations, when the manipulator 14 operates in the semi-autonomous mode, the manipulator 14 is capable of moving the tool 20 free of user assistance. Free of user assistance may mean that a user does not physically contact the tool 20 to move the tool 20. Instead, the user may use some form of remote control to control starting and stopping of movement. For example, the user may hold down a button of the remote control to start movement of the tool 20 and release the button to stop movement of the tool 20. The system 10 may also operate in a fully automated mode wherein the manipulator 14 is capable of moving the tool 20 free of user assistance or override.

The system 10 may also operate in a guided-manual mode to remove the remaining sub-volumes of bone, or for other purposes. An example of operation in the guided-manual mode is also described in U.S. Pat. Publication No. 2020/0281676, incorporated above. In this mode, aspects of control used in both the manual mode and the semi-autonomous mode are utilized. For example, forces and torques applied by the user are detected by the force/torque sensor S to determine an external force Fₑₓₜ. The external force Fₑₓₜ may comprise other forces and torques, aside from those applied by the user, such as gravity-compensating forces, backdrive forces, and the like, as described in U.S. Pat. No. 9,119,655, incorporated above. Thus, the user-applied forces and torques at least partially define the external force Fₑₓₜ, and in some cases, may fully define the external force Fₑₓₜ. Additionally, in the guided-manual mode, the system 10 utilizes a milling path (or other tool path) generated by the path generator 68 to help guide movement of the tool 20 along the milling path.

### II. Techniques for Guided Placement of the Manipulator

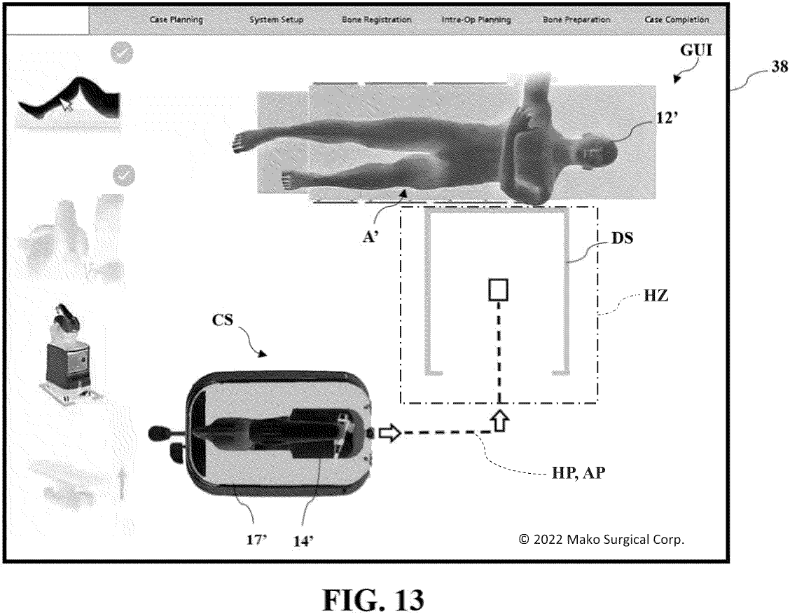

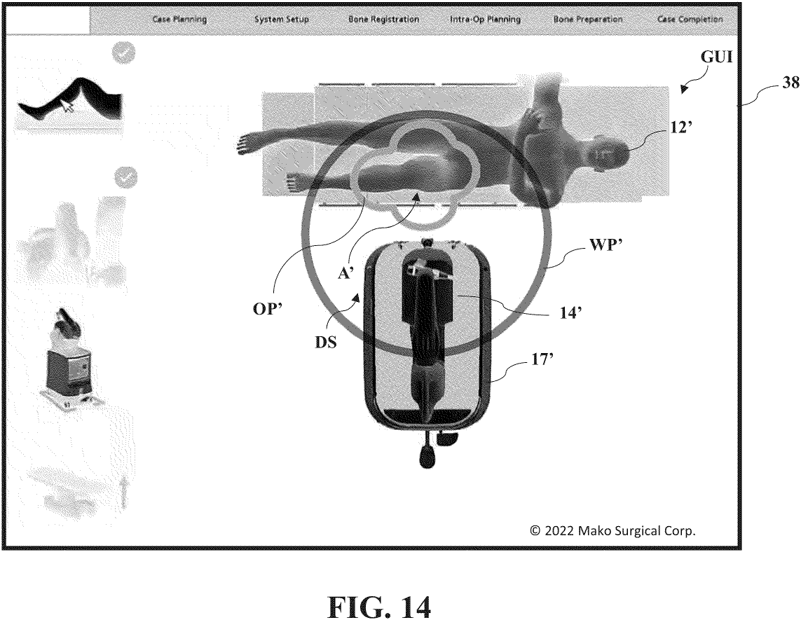

Described in this section are systems, methods, guidance systems, and computer-implemented techniques to guide placement of the manipulator 14 to a desired state DS in the operating room (See FIGS. 13 and 14). The techniques can involve the navigation system 32 tracking the patient anatomy (A) and tracking a current state CS of the manipulator 14 relative to the anatomy (A). One or more controllers, including but not limited to the control system 60, navigation controller 36, and manipulator controller 26, obtain workspace parameters of the manipulator 14. The one or more controllers 60, 36, 26 capture, from the localizer 44, the states of the anatomy (A) in response to movement of the anatomy (A). The one or more controllers 60, 36, 26 determine operative parameters of the anatomy (A) based on the captured state of the anatomy (A). The one or more controllers 60, 36, 26 determine the desired state DS for the manipulator 14 based on evaluation of the workspace parameters and operative parameters and guide placement of the manipulator 14 from the current state CS to the desired state DS.

### A. Manipulator and Placement Thereof

The manipulator 14 is placed from the current state CS to the desired state DS. For the numerous examples described herein, the current state CS of the manipulator 14 can be a current location of the base 16 of the manipulator 14 in the operating room, a current pose of the robotic arm of the manipulator 14, and/or both a current location of the base 16 and current pose of the robotic arm 14. For the numerous examples described herein, the desired state DS of the manipulator 14 similarly can be a desired location of the base 16 of the manipulator 14 in the operating room, a desired pose of the robotic arm of the manipulator 14, and/or both a desired location of the base 16 and desired pose of the robotic arm of the manipulator 14.

Placement of the manipulator 14 to the desired state DS can involve placement relative to the patient 12 anatomy (A). Placement of the robotic manipulator 14 to the desired state DS can alternatively involve placement relative to any surgical object other than the patient 12, such as the surgical table. Placement of the robotic manipulator 14 to the desired state DS can involve physically moving the base 16 of the manipulator 14 from its current location to the desired location adjacent to the patient 12 or patient table. Additionally, or alternatively, placement of the robotic manipulator 14 to the desired state DS can involve physically controlling the joints (J) of the manipulator 14 to change from a current state or pose to a desired state or pose. Here, the robotic manipulator 14 may be within range of the patient or not. The current pose can be a “ready” pose, a stowed pose, a transportation or shipping pose, or any other type of pose. The desired pose can also be a “ready” pose or a modification to the “ready” pose, or any other pose.

The desired state DS of the manipulator 14 can be a pre-operative state, i.e., a state that the manipulator 14 should be in before surgery. The desired state DS can also be an intra-operative state, wherein the patient is undergoing surgery, but the surgical procedure may be temporarily paused to provide time to change the current state CS to the desired state DS. In either instance, it should be understood that the techniques described herein are intended to provide the desired state DS to initially “set-up” the manipulator 14 for surgery and/or to reconfigure the manipulator 14 during a pause in surgery.

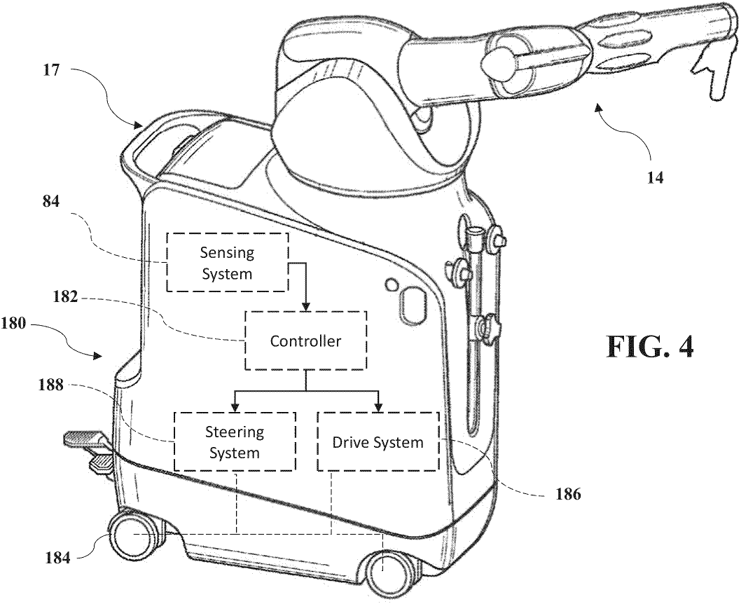

The techniques described herein can be utilized with any manipulator 14 in the operating room. In one implementation, the manipulator 14 has a moveable cart 17, for example, as shown in FIGS. 1 and 4. The cart 17 may be specifically to support the manipulator 14. Alternatively, the cart 17 could support additional devices, such as imaging devices, monitors or displays, and the like. In other configurations, the manipulator 14 can be mounted to a moveable imaging device or gantry, such as a CT, X-Ray, or Fluoroscopy imaging device or scanner. The imaging device can serve as a moveable cart. One example of a manipulator 14 that can be utilized with an imaging device can be like that described in U.S. Pat. No. 11,103,990, entitled “System and Method for Mounting a Robotic Arm in a Surgical Robotic System” the contents of which are hereby incorporated by reference in its entirety. In these instances, placement of the robotic manipulator 14 to the desired state DS can involve physically moving the manipulator 14 along the floor of the operating room by any moving support unit, such as the cart 17 or imaging device. Additionally, or alternativity, placement of the robotic manipulator 14 to the desired state DS can involve changing the pose of the manipulator 14 to the desired state DS. The change in pose can occur at any location within the operating room, i.e., at the current or desired location or anywhere in-between.

In other implementations, the manipulator 14 can be mounted to a ceiling unit or moveable overhead unit, such as a surgical boom. The surgical boom may provide an adjustment system or track to enable the manipulator 14 to move relative to the surgical boom. Here, the manipulator 14 may be placed in the desired state DS by being moved along a track or lowered by a lowering mechanism provided by the surgical boom. Additionally, or alternativity, placement of the robotic manipulator 14 to the desired state DS can involve changing the pose of the manipulator 14 to the desired state DS. The change in pose can occur at any location relative to the surgical boom, i.e., at the current or desired location or anywhere in-between.

The manipulator 14 can also be hand-held and supported freely by the hand of a user against the force of gravity. The hand-held manipulator 14 can be releasably attachable to a support arm (base) that can be passively or actively adjustable and lockable to a pose. Here, the current state CS of the manipulator 14 can be its current state at which the user is freely holding the manipulator 14 in the operating room or its current state at which the hand-held manipulator 14 is attached to the support arm. The current state CS could be how the manipulator 14 is currently positioned and/or oriented by the surgeon or could be a current kinematic pose of the hand-held manipulator 14 in instances where the manipulator 14 has actuatable joints (J). The desired state DS of the hand-held manipulator 14 can be a desired state at which the user should freely hold the manipulator 14 relative to the patient 12 or a desired state at which the hand-held manipulator 14 should be located while attached to the support arm. The desired state DS could be how the manipulator 14 should be positioned and/or oriented by the surgeon or could be a desired kinematic pose of the hand-held manipulator 14 in instances where the manipulator 14 has actuatable joints (J). Here, the hand-held manipulator 14 joints (J) can be actuated to change from the current pose to the desired pose. In other implementations, the manipulator 14 can be table mounted, patient mounted or both table and patient mounted. Any other type of manipulator 14 is contemplated.

Furthermore, in some implementations, the techniques described herein can be expanded to additionally guide placement of the anatomy (A) from a current state to a desired state of the anatomy (A). The desired state of the anatomy (A) can be a location and/or pose of the anatomy (A) relative to any reference point, such as the localizer 44, the surgical table, the manipulator 14, the end effector 22, tool 20 and/or TCP and the like. Determination of the desired state of the anatomy (A) can be implemented using any of the techniques, sources, inputs, and methodologies describe herein with reference to the desired state DS of the manipulator 14.

### B. Tracking States of Manipulator and Patient

The one or more controllers 60, 36, 26 can track the state (base location and/or arm pose) of the manipulator 14, the anatomy (A), and the relationship between the manipulator 14 and anatomy (A) using various tracking techniques.

In one example, the one or more controllers 60, 36, 26 do so using any of the described trackers, such as the base tracker 52B, end effector tracker 52A and patient trackers 54, 56. To track the location of the manipulator 14, the one or more controllers 60, 36, 26 compare the location of the base tracker 52B and/or end effector tracker 52A relative to the patient trackers 54, 56 in the localizer coordinate system LCLZ.

Additionally, or alternatively, the one or more controllers 60, 36, 26 can utilize kinematic data from the manipulator 14 to determine the state (location and/or pose) of the manipulator 14. This kinematic data can be the state of the tool 20, e.g., relative to the manipulator coordinate system MNPL or relative to the base 16. In one instance, the kinematic data may be obtained from the manipulator controller 60 applying a forward kinematic calculation to values acquired from the joint encoders 19. Thus, the state of the tool 20 can be determined relative to the manipulator coordinate system MNPL without intervention from the navigation system 32 or obtained irrespective of any measurements from the navigation system 32. In some instances, as will be described below, the anatomy (A) is moved by an anatomical manipulator (limb holder), and kinematic data can be similarly obtained from such manipulators to determine the state of the anatomy (A).

The navigation system 32 can also fuse kinematic data with localization data. The navigation system 32 can also use various transforms to discern the relationship between the localizer 44, manipulator 14 and anatomy (A). These transforms can be like those described in U.S. Pat. Application Publication No. 2020/0237457, entitled “Techniques for Detecting Errors or Loss of Accuracy in a Surgical Robotic System”, the entire contents of which are hereby incorporated by reference.

The one or more controllers 60, 36, 26 can use any alternative localization modality described above (e.g., RF, electromagnetic, machine vision, ultrasound) and can use hybrid modalities. For instance, the one or more controllers 60, 36, 26 can track the anatomy (A) using ultrasound tracking and track the state of the manipulator 14 using optical tracking. Alternatively, the one or more controllers 60, 36, 26 can track the state of both the anatomy (A) and the manipulator 14 using machine vision or computer vision tracking, without the need for any tracking devices fixed to the anatomy (A) or manipulator 14. Other examples are contemplated.

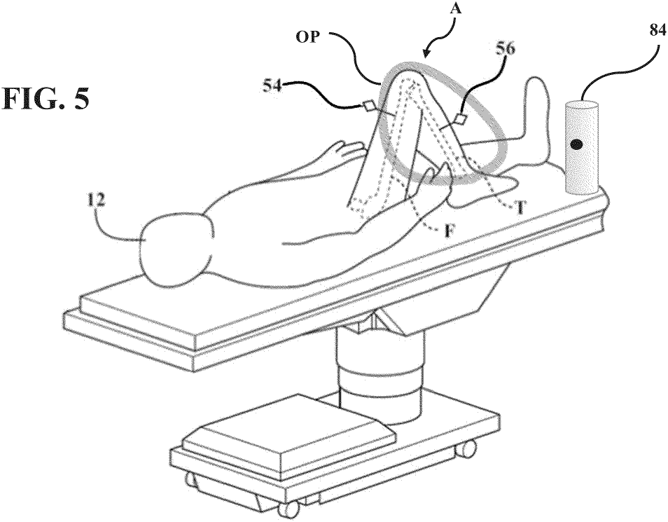

In another implementation, as shown in FIGS. 4 and 5, the anatomy (A), manipulator 14 and/or cart 17 can optionally or additionally be equipped with, or interface with, one or more sensing systems 84 that can include devices for sensing the surroundings and states of the anatomy (A), manipulator 14, cart 17 or other objects in the operating room. The sensing systems 84 can include proximity sensors, video cameras, range or distance finders LIDAR sensors, ultrasonic sensors, inertial sensors, radar sensors, and any combination thereof. The sensing system(s) 84 can be coupled to the navigation system and localizer 44. The one or more controllers 60, 36, 26 can communicate with the sensing system 84 wirelessly or through wired connection to detect states and perform actions, as will be described below. In one implementation, the sensing system 84 can be temporarily positioned in the operating room for the techniques described herein and removed afterwards. In other examples, the sensing system 84 is coupled to one or more objects in the operating room. For the manipulator 14, as shown in FIG. 4, the sensing system 84 can be integrated into the manipulator 14 or cart 17. The sensing system 84 of the manipulator 14 or cart 17 can detect the states of the manipulator 14, cart 17 and the anatomy (A). For the anatomy (A), as shown in FIG. 5, the sensing system 84 can be placed on or adjacent to the patient 12, for example, adjacent to the surgical table. Multiple sensing system 84 can be utilized and the sensing systems 84 can detect each other to define relative relationships between each other and their respective objects, e.g., patient 12 and manipulator 14.

In other implementations, the navigation system 32, including the localizer 44, can be incorporated with the cart 17 for movement therewith and for detecting states of objects, including the cart 17, and the anatomy (A).

### 1. The Patient Anatomy

As introduced, the techniques described herein can utilize the states of the anatomy (A) to provide input into guiding placement of the manipulator 14 to the desired state. The state of the anatomy (A) can be any number of positions and/or orientations of the anatomy (A).

As used herein the patient anatomy (A) refers to a region of the anatomy of the patient 12 which is being subjected to or will be subject to a surgical procedure. These anatomical regions can include, but are not limited, to one or more of the following: a single bone, an anatomical joint, two or more bones forming a joint, a leg, a femur, a tibia, a hip, a pelvis, a knee, a shoulder, a humerus, a scapula, a spine, a vertebra or vertebrae, a skull or cranial region, an ankle or ankle bones, an organ, soft tissue, and the like.

In one example, the anatomy (A) can be the general anatomical region that supports or includes the actual site of surgery. Here, the anatomy (A) can be the external region of the patient 12 and not necessarily the internal structures of the patient 12. For instance, in a total or partial knee replacement or revision procedure, the anatomy (A) can be the external knee region, or the knee region and the external leg of the patient 12. In a hip replacement or revision procedure, the anatomy (A) can be the external hip region, or the hip region and external leg of the patient 12.

Additionally, or alternatively, the anatomy (A) can be the specific internal anatomical region which is, or will be, manipulated by the manipulator 14 during surgery. In other words, the anatomy (A) can be the surgical site SS. For instance, in a total or partial knee procedure, the anatomy (A) can be the internal joint region, or the femur and/or tibia bones and any associated internal soft tissue surrounding the area. In a hip procedure, the anatomy (A) can be the pelvic bone and/or the femur bone of the patient 12 and any associated internal soft tissue surrounding the area. The anatomy (A) can also include both the external and internal regions of the patient 12.

Also, the anatomy (A) can be a closed (i.e., before any incision). Mainly, the techniques described herein can be utilized to place the manipulator 14 before the surgical procedure occurs. In other examples, the anatomy (A) may not be incised during surgery. Alternatively, the anatomy (A) can include, but is not necessarily limited to, the region of the anatomy that is surgically opened by an incision. The anatomy (A) can also be accessed percutaneously, subcutaneously, and/or in a minimally invasive manner.

### C. Example Methodology to Guide Placement of Manipulator Relative to Anatomy

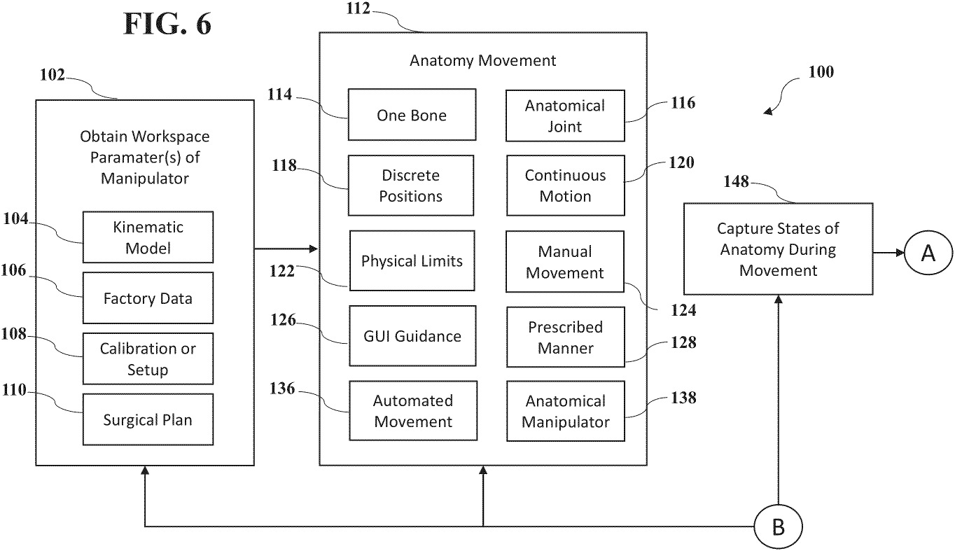

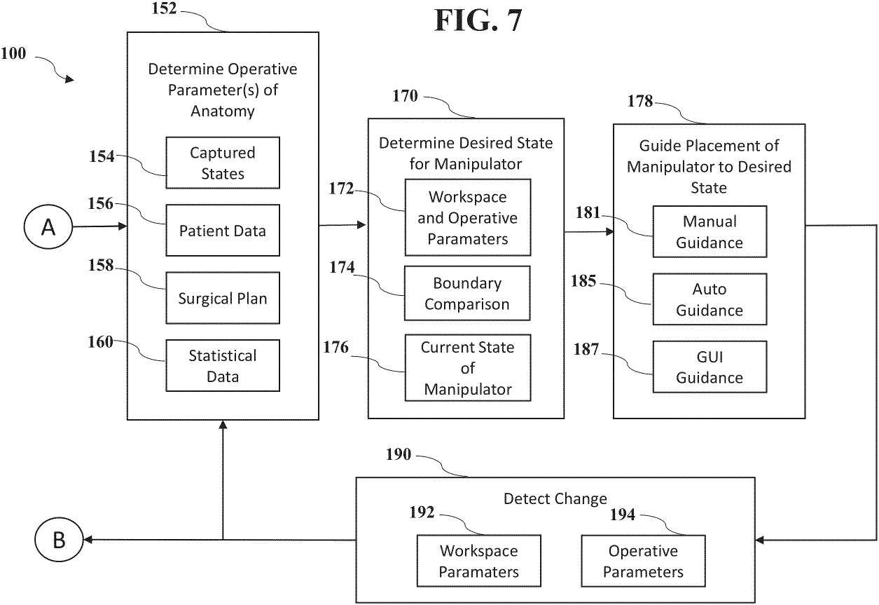

Referring now to FIGS. 6 and 7, a block diagram and flowchart is illustrated of one implementation or method 100 of guiding placement of the manipulator 14 relative to the anatomy (A). The steps of the example method 100 include: step 102 of obtaining the workspace parameters of the manipulator 14; step 112 of moving the patient anatomy; step 148 of capturing states of the anatomy (A) during movement; step 152 of determining operative parameters of the anatomy (A); step 170 of determining a desired state DS for the manipulator 14 based on evaluation of the workspace parameters and operative parameters; step 178 of guiding placement of the manipulator 14 from the current state CS to the desired state DS; and step 190 of detecting changes and taking action to re-evaluate any step of the method 100.

The order of these steps can differ from what is shown in FIGS. 6 and 7 and should not be limited to the exact order shown. Furthermore, some steps may be optional depending on the implementation. Hence, not all steps may be required. The method 100 can be implemented as a system 10, a guidance system (implemented by the navigation system 32), and/or a non-transitory computer-readable medium or software program product. The method 100 is described with a focus on the anatomy (A), however, the method 100 can be implemented to guide placement of the manipulator 14 to any other surgical object. The steps of the method 100 will be described in detail below.

### 1. Workspace Parameters of Manipulator

In FIG. 6, the one or more controllers 60, 36, 26 obtain workspace parameters WP of the manipulator 14, at step 102. As used herein “workspace parameter” defines parameters of how the manipulator 14 moves in space. The workspace parameters WP can include how the joints J, end effector 22, tool 20 and/or TCP may move in space. The workspace parameters WP can also define limits on motion of the manipulator 14 or its various components.

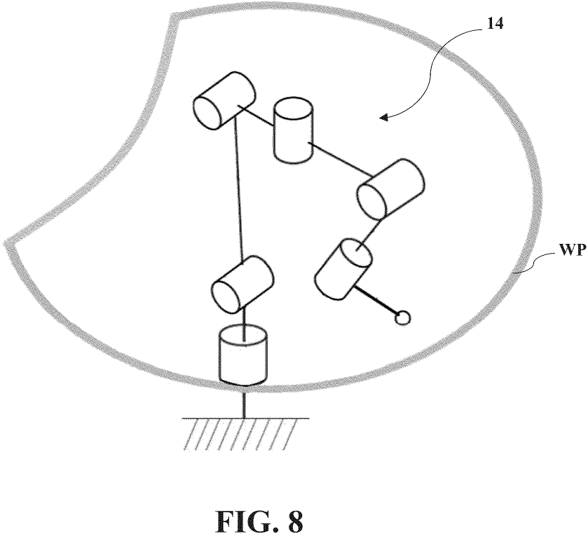

FIG. 8 provides a graphical illustration of an example of workspace parameters WP shown relative to the manipulator 14. The workspace parameters WP, in one example, include a workspace boundary or envelope. The workspace parameters WP can define a space, which can be a volume or area. The volume or area can be an open or closed geometry. Depending on the configuration of the manipulator 14, the workspace parameters WP can define a Cartesian, cylindrical, spherical, and/or articulated space or any other partial version thereof. The workspace parameters WP can be an actual or physical space and/or data defining this space. The workspace parameters WP can be visualized by the GUI or can be defined by data that is hidden to the user.

In one implementation, the workspace parameters WP can be defined by a space swept out by the end effector 22, tool 20 and/or TCP as the manipulator 14 executes kinematic motions. In another instance, the workspace parameters WP can be defined by the total space swept in all possible kinematic motions. The workspace parameters WP can alternatively be defined by the total space swept in some kinematic motions. For instance, the manipulator 14 may be limited to certain degrees of freedom for specific types of surgery, certain steps of a procedure, and/or certain tools of the procedure. In another implementation, the workspace parameters WP are defined by a reachable workspace whereby the end effector 22, tool 20 and/or TCP is capable of reaching each point within the reachable workspace in at least one orientation. In another example, the workspace parameters WP are a dexterous workspace whereby the end effector 22, tool 20 and/or TCP is capable of reaching some or all points in some or all orientations. The workspace parameters WP can consider where the joints J or links 18 of the manipulator 14 move, including the external surface of the links 18. For instance, the workspace parameters WP can include a 3D joint workspace envelope, which includes the space swept out by some or all of the joints J and links 18 as the manipulator 14 executes kinematic motions. Alternatively, or additionally, the workspace parameters WP can be a 2D or 3D functional workspace, which can be a subset of the 3D joint workspace. The functional workspace can be limited to motion of the end effector 22, tool 20 and/or TCP. The workspace parameters WP can consider joint J limits, whether such limits are virtual or physical. The workspace parameters WP can be defined using any combination of the data described herein, or using other data not specifically defined herein. Furthermore, any of the described data can be derived from physical or simulated analysis of the manipulator 14.

At box 104, the workspace parameters WP can include the kinematic data of the manipulator 14. For instance, this kinematic data can define the joints J or links 18, the type of joints J or links 18 (e.g., revolute, prismatic), the relationship between the joints J or links 18, the length or external size of the joints J or links 18, encoder 19 parameters or data, data identifying singularities that may occur from movement of two or more joints J, degrees-of-freedom or constraint parameters of the manipulator 14, geometry or data related to the base 16 or manipulator coordinate system MNPL, and geometry or data related to the end effector 22, tool 20, or TCP, and the like. The kinematic data can be a kinematic model, such as one that defines the motion of the manipulator 14 without regard to forces/torques that cause such motion. The kinematic data can additionally or alternatively include a dynamic model which defines the relation between applied forces/torques resulting from motion of the manipulator 14. These models can be forward or inverse models.

At box 106, the workspace parameters WP can be derived from or include factory data. The factory data may be determined and stored during manufacture or assembly of the manipulator 14. The factory data can include any of the workspace parameters WP described herein, such as kinematic data.

At box 108, the workspace parameters WP can be derived from or include calibration data or data from setting up the manipulator 14. The calibration data can be factory data or can be determined on-site or in the operating room. The calibration data can define a current state CS of the manipulator 14, where the current state CS may differ from its original factory state. The calibration data can also compare any expected and actual values of the manipulator 14. For example, calibration data may be derived from comparing relative position of the links 18 or joints J, actual and reported joint torques/displacements/positions, joint angle offsets, joint lengths, joint stiffness, joint compliance, and the like. Calibration can be performed by the manipulator 14 going through a physical test, such as a predetermined movement whereby the one or more controllers 60, 36, 26 compare the parameters. Calibration can be performed by an external device, such as a laser tracker that tracks the manipulator 14, for example, using an end effector tracker that reflects laser signals. Calibration can be performed using a telescoping bar connected between a fixed reference datum and the TCP, whereby the length of the telescoping bar is compared to predetermined data. Any other type of robotic calibration technique is contemplated to derive workspace parameters WP of the manipulator 14.

At box 110, the workspace parameters WP can be derived from or include surgical plan data. The surgical plan data can define or modify the workspace parameters WP. In one example, the surgical plan data may limit the workspace parameters WP of the manipulator 14. The surgical plan data can be determined by a surgeon or can be a specified or predetermined set of parameters for given a situation, condition, or procedure. The surgical plan data can also be patient-specific and/or based on generic surgical data, for instance from a statistical population of manipulators, patients, or procedures. The surgical plan data can include the type of procedure, the type or size of implant, the surgical approach (surgical access direction or plan) of the procedure, parameters for different steps of the procedure, the types and geometries of tools of the procedure, when such tools are planned to be used during the procedure, parameters of the anatomy, including planned treatment region, cut planes and cut poses, target axes, resection volumes, or the like. The surgical plan data can include the virtual boundaries generated by the boundary generator 66 where such virtual boundaries constrain movement of the manipulator 14. The surgical plan data can include how many virtual boundaries, the location of these virtual boundaries, and the reactive force imparted by collision of these virtual boundaries. The surgical plan data can include the tool paths generated by the path generator 68 where such tool paths constrain movement of the manipulator 14 or TCP. The surgical plan data can include the feed rates which limit or define the speed along which the manipulator 14 moves the energy applicator or TCP along any tool path. The surgical plan data can include the cutting speeds or rates (e.g., rotational speed, oscillating speed) which limit or define the speed of, or energy provided by, the energy applicator. The surgical plan data can include bone mineral density values which can affect how the manipulator 14 moves or applies force. The surgical plan data can include preferred tool orientation data or preferred manipulator pose data.

Workspace parameters WP can also be derived from limitations or preferences related to the manipulator trackers 52A, 52B and their relationship to the navigation system 32. For instance, the workspace parameters WP could define line-of-sight conditions related to visibility of the manipulator trackers 52A, 52B relative to the localizer 44. This data could define the range of motion of the manipulator 14 that is preferred to optimize visibility and/or limit the manipulator 14 from assuming certain poses that may obstruct tracker visibility. Other types of surgical plan data are contemplated.

Any of the described surgical plan data examples can be specific to any procedure or any single step of a procedure. The workspace parameters WP can be derived using any combination of the sources described herein, or using other sources not specifically defined herein. Also, the one or more controllers 60, 36, 26 can obtain the workspace parameters WP from any suitable source and in any suitable manner. For instance, the workspace parameters WP can be stored locally on a non-transitory memory that is accessible to the one or more controllers 60, 36, 26. The memory can be located anywhere, including on the manipulator 14, on the cart 17, on the navigation system 32, on a remote server or cloud, or using a memory drive that is manually provided to the one or more controllers 60, 36, 26. The workspace parameters WP can be transmitted to the one or more controllers 60, 36, 26 using a wired or wireless connection. The workspace parameters WP can be obtained preoperatively and/or intraoperatively. In some instances, the workspace parameters WP can be derived from artificial intelligence and/or machine learning algorithms which take into consideration post-operative manipulator 14 or surgical data.

### 2. Patient Anatomy Movement

In FIG. 6, the patient anatomy (A) is moved at step 112. In one implementation, movement of the anatomy (A) is performed to provide an input into determining the desired state DS of the manipulator 14. In one instance, the anatomy (A) is moved after the patient 12 is placed on the surgical table in the operating room and before the procedure begins. In another implementation, the anatomy (A) is moved in response to an updated desired state DS of the manipulator 14 whereby the surgical procedure can be paused to reconfigure the manipulator 14. Several types of anatomy (A) movement will be described. The source of the anatomy (A) movement can vary according to different implementations. Any of these implementations can be combined in part or in whole.

The anatomy (A) that is moved can be the like that described above, e.g., any external and/or internal part of the anatomy (A). In one example, as shown at 114, the anatomy (A) can be a single bone. In another example, as shown at 116, the anatomy (A) can be two or more bones, or an anatomical joint of the patient 12, such as a knee, hip, or shoulder joint.

Movement of the anatomy (A) can be any positioning and/or orienting of the anatomy (A) according to any number of degrees of freedom and according to any suitable manner. For instance, the anatomy (A) can be translated in any direction parallel to or perpendicular to a plane of the surgical table. When the anatomy (A) is an anatomical joint, the joint can be flexed, extended, tilted in one or more directions, rotated in one or more directions, or any combination thereof. Movement of the anatomy (A) can also involve changing a general position of the patient 12 on the surgical table, changing a pose or height of the surgical table, or the like.

In one implementation, as shown at 118, the anatomy (A) can be moved to one or more discrete positions. These discrete positions can be predefined, guided by the system 10, and/or defined by the staff during movement. A discrete position may be for instance movement of the anatomy (A) to a specific angle or pose.

Additionally, or alternatively, as shown at 120, the anatomy (A) can be moved according to continuous motion. A continuous motion may be, for instance, a continuous motion of a range of motion, such as flexion and extension of the anatomy (A). Here, continuous motion is contrasted with discrete positions in that continuous motion involves moving the anatomy (A) along a range of poses, rather than at one or more single poses. The continuous motion can also be predefined, guided by the system 10, and/or defined by the staff during movement.

In another implementation, as shown at 122, the anatomy (A) can be moved to its physical limits. For example, where the anatomy (A) is an anatomical joint, the joint can be flexed and extended to the flexion/extension limits of the joint. The joint can be tilted or rotated to the tilt or rotational limits of the joint. The physical limits of the anatomy (A) can be predefined, guided by the system 10, and/or defined by the staff during movement by the staff, for example, as the staff feel resistance of the anatomy (A) at the limits. Other ways of moving the anatomy (A) are contemplated and described below.

### i. Manual Techniques for Anatomy Movement

In one implementation, as shown at 124, the source of the anatomy (A) movement is a manual source of movement. For instance, the surgeon or other staff may physically grasp the anatomy (A) and reposition and/or re-orient the anatomy (A) in any suitable manner, such as the movements described above. Alternatively, or additionally, manual movement may involve a staff member changing the height, position, or orientation of the surgical table. Additionally, any types of manual positioning tools or fixtures may be utilized to manual move the anatomy (A). For instance, a staff member may manually move a limb holder or brace that supports the anatomy (A). Other types of manual movement are contemplated.

### ii. Guided Movement of Anatomy Using Graphical User Interface

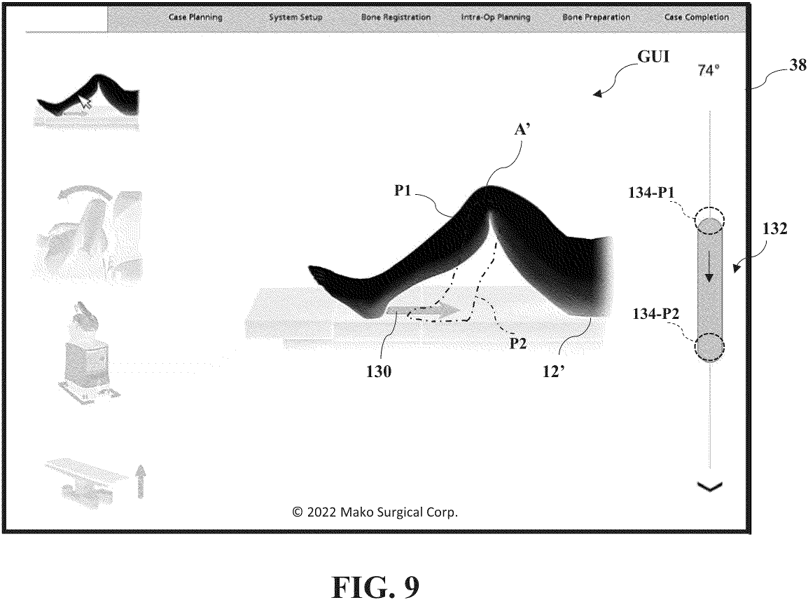

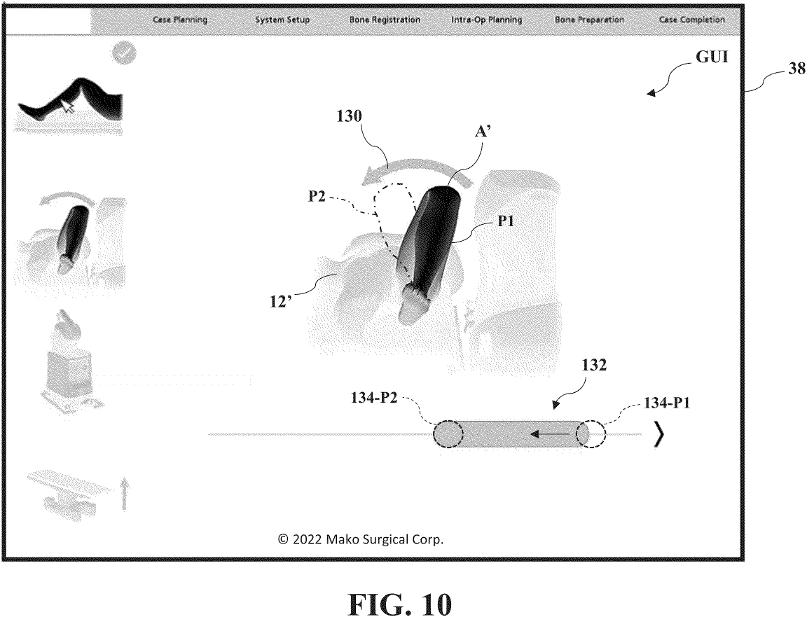

In another implementation, as shown at 126, the movement of the anatomy (A) can be guided by the clinical application 80, user interface UI, and/or GUI (shown in FIGS. 9 and 10). Here, the one or more controllers 60, 36, 26 can implement the GUI that is displayed on any one or more of the display devices 38. The GUI can display an actual image or video of the patient anatomy (A) and the patient 12 (or part of the patient). Alternatively, as shown in FIGS. 9 and 10, the GUI can display a graphical representation of the anatomy and the patient (or part of the patient), identified respectively by reference numerals 12′ and (A′). The GUI can provide feedback about how the anatomy (A) is moving in real-time by updating the actual image or graphical representation of the anatomy (A′) to reflect real-world movement of the anatomy (A). Alternatively, or additionally, the GUI can display suggestions, notification, or alerts about how or how-not-to move the anatomy (A).

In one implementation, as shown at 128, the GUI can be utilized to guide movement of the anatomy (A) according to a prescribed or recommended manner presented to the user by the GUI. The prescribed/recommended manner can be defined according to surgical plan data or patient-specific data. The prescribed/recommended manner can include one or multiple steps that are directed by the GUI. In one example, the anatomy (A) is a knee and the prescribed/recommended manner is implemented by the GUI instructing or prompting the user to flex or extend the knee or leg of the patient 12 (as shown in FIG. 9) and, in another step, instructing or prompting the user to tilt the knee or leg of the patient 12 (as shown in FIG. 10). In one implementation, one step must successfully be completed before the GUI allows the software to proceed to the subsequent step. Alternatively, the steps can be performed in any order and re-performed at any suitable time should the prescribed movement be unsuccessful. In one example, the prescribed/recommended movement for a knee is to flex the knee from 95 to 110 degrees. Furthermore, the prescribed/recommended movement for the tilt of the knee may depend on the outcome of the flexion angle. Therefore, the prescribed/recommended manner can be determined by the one or more controllers 60, 36, 26 on-the-fly.

The GUI is configured to provide feedback to the user during movement according to the prescribed/recommended manner. The feedback can be visual, audible, haptic, or any combination thereof. The GUI can instruct the user using instructional indicia 130 (e.g., icons, text, animations, videos, or symbols), such as the arrows shown in FIGS. 9 and 10. The arrow in FIG. 9 instructs the user to flex the knee or leg in a certain direction and the arrow in FIG. 10 instructs the user to tilt the knee or leg in a certain direction. The instructional indicia 130 can indicate a direction the user should move the anatomy (A), a pose to which the anatomy (A) should be moved, and/or instructions about how much more movement is needed to get from the current position of the anatomy (A) to a prescribed/recommended position. For example, the GUI can guide the user to “flex the knee an additional 15 degrees” or “tilt the knee an additional 20 degrees”. The instructional indicia 130 can dynamically change depending on how the anatomy (A) is moved, was moved, or should be moved. For example, the arrow may change for flexion or extension directions.

In one example, as shown in FIGS. 9 and 10, the GUI can implement the prescribed/recommended manner by displaying a target indicator 132 guiding the user to move the anatomy (A) to a target pose and/or to move the anatomy (A) within a target range. In FIGS. 9 and 10, the target indicator 132 is implemented as a scrolling bar which shows a desired range of motion of the anatomy (A) to be captured. The desired range can be shown as a geometric object (e.g., elongated oval) that extends along the axis of the scrolling bar. Other ways of showing the desired range are contemplated. The GUI can also provide a moveable indicator 134 that is configured to move in response to corresponding movement of the anatomy (A). The moveable indicator 134 can move relative to the target indicator 132 (e.g., target position and/or target range) to provide visual guidance on relative positioning between the anatomy (A) and the target indicator 132. In FIGS. 9 and 10, the anatomy (A) is moved from a first position P1 to a second position P2. When the anatomy (A) is in the first position P1, the moveable indicator is shown at a location on the scrolling bar at 134-P1. When the anatomy (A) is in the second position P2, the moveable indicator is shown at a second location on the scrolling bar at 134-P2. Between the first and second positions P1, P2, the movable indicator 134 will move along the scrolling bar in the direction of the arrow, and vice-versa. This indicator 134 movement along the scrolling bar will correspond to the actual movement of the anatomy (A). By sweeping the movable indicator 134 in this manner, the GUI enables the user to see how much the anatomy (A) should be moved and in what direction to enable a full sweep of the target indicator 132. Techniques described here can be similarly utilized where the target indicator 132 is one or more discrete locations, rather than a range as shown. A real-time angle of flexion or extension (e.g., 74 degrees) or tilt of the anatomy (A) can be automatically updated and shown by the GUI, as shown in FIG. 9.

The target indicator 132 and moveable indicator 134 can be implemented in various manners other than the manner shown in FIGS. 9 and 10. In another example, the target indicator 132 can be graphically shown on the GUI as one or more anatomical pose(s) and/or an anatomical range of motion of the anatomy (A). This could be, for example, an anatomical envelope of the range of motion or an outline of where the anatomy (A) should be moved. This outline can be in the actual shape of the anatomy (A) or a generic shape of the anatomy (A). For instance, instead of P2 and its respective outline in FIGS. 9 and 10 representing the second position to where the anatomy (A) was actually moved, P2 and its respective outline could be the target indicator 132, e.g., indicating where the GUI desires the anatomy (A) to be moved. Meanwhile, in this example, the moveable indicator 134 can be the actual imagery of the anatomy (A) or graphical representation of the anatomy (A′), such as that shown on the GUI. In other words, the user can be guided by comparing, on the GUI, the state of the representation of the anatomy (A) to the state of the target indicator 132 in order to sweep the range or arrive at the desired target pose.

Once the GUI detects alignment to, or sweeping of, the target indicator 132 by the movable indicator 134, the GUI can provide feedback to the user that an acceptable target motion has been achieved, for example, to enable downstream steps, such as determining the desired state DS of the manipulator 14. In such instances, a target confirmation indicator can be triggered to provide the user with feedback. The target confirmation indicator can be a change to the color of target indicator 132 (e.g., to green) or a flashing of the target indicator 132 to provide this confirmation. Alternatively, the target confirmation indicator can be icons, text, animations, videos, or symbols that convey confirmation of target motion. Once the movement capture is successful, the GUI may advance to the next step of the prescribed/recommended motion, if applicable. The GUI can be used with in this manner described for any of the techniques described herein for moving the anatomy (A).

### iii. Automated Techniques for Anatomy Movement

In another implementation, as shown at 136 in FIG. 6, the source of the anatomy (A) movement is an automated or semi-automated source of movement. The automated or semi-automated movement may be automatically determined by the system or configured by a surgeon or staff. Parameters of the automated movement can be like any of those described above, e.g., discrete positions, continuous motions, prescribed or recommended motions, target pose(s), target range(s), or the like.

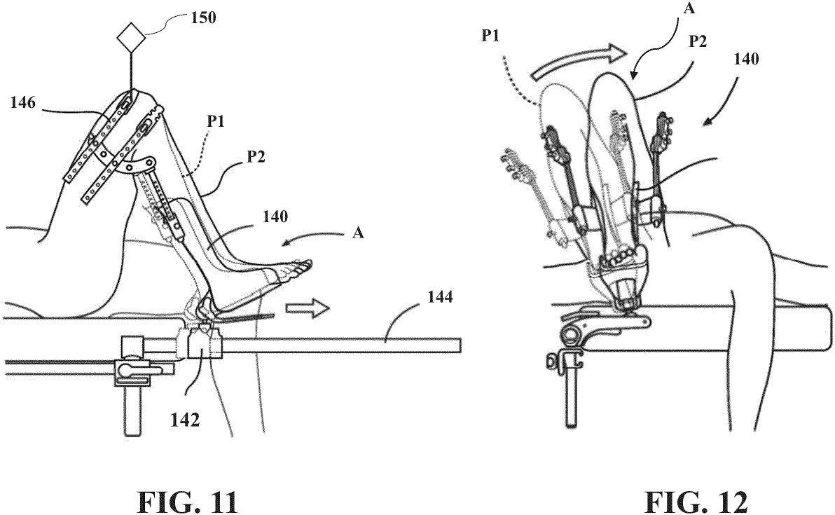

In one implementation, as shown in the example of FIGS. 11 and 12, an anatomical manipulator 140 may be coupled to the anatomy (A) to provide automated motion at or between different poses (for example, shown as P1 and P2). The automated manipulator 140 may reposition and/or re-orient the anatomy (A) in any suitable manner, such as the movements described above. The anatomical manipulator 140 can be a motorized joint positioner that couples to and holds the limb of the patient (e.g., a motorized limb holder). In FIGS. 10 and 11, for example, the anatomy (A) supported by the anatomical manipulator 140 is a leg and knee. The anatomical manipulator 140 can include mechanisms for extending or flexing the anatomy (A). The anatomical manipulator 140 in this example is supported by a sled 142, which moves along a support bar 144. The automated movement may involve moving the sled 142 along the support bar 144 to accomplish a desired movement the anatomy (A). The direction of the automated movement of the sled 142 in FIG. 11 is illustrated by an arrow for simplicity. As shown in FIG. 12, the anatomical manipulator 140 may also rotate or tilt the anatomy (A) medially (toward or away from a centerline of the patient). Furthermore, as shown in FIG. 11, securing mechanisms 146 can be used for securing the other portions of the anatomy (A). The anatomical manipulator 140 can be locked in any given position.

Of course, the anatomical manipulator 140 may have various other configurations and may be manipulated in various other ways. In another implementation, the manipulator 14 itself is used as the anatomical manipulator 140. For instance, a limb holder end effector could be attachable to the manipulator 14 to enable movement of the anatomy (A). Afterwards, the limb holder end effector can be swapped for the end effector 22 to manipulate tissue during the procedure. In this example, the one or more controllers 60, 36, 26 can control the manipulator 14 to move the limb holder end effector in any suitable automated fashion as described. In some instances, the force/torque sensor S can detect forces/torques that are indicative of target poses or target ranges of the anatomy (A). The one or more controllers 60, 36, 26 can detect these forces/torques to identify that the anatomy (A) has been moved as prescribed or recommended. In other implementation, the one or more controllers 60, 36, 26 can capture and analyze joint encoder data and/or joint motor torque to determine the anatomy (A) has been moved as prescribed or recommended. The anatomical manipulator 140 can be like those described in US Pat. Application Pub. No. 20190262203, entitled “Motorized Joint Positioner” or like US Pat. No. 10390737, entitled “System and Method of Controlling a Robotic System for Manipulating Anatomy of a Patient During a Surgical Procedure” the contents of both of which are hereby by reference in their entirety.

Alternatively, or additionally, an automated surgical table may be provided which can automatically change the height, position, or orientation of the surgical table to effect automated movement of the anatomy (A). Other types of automated devices or techniques are possible.

### 3. Capture States of Anatomy During Movement

With reference to FIG. 6, the method 100 includes the step 148 of the one or more controllers 60, 36, 26 capturing states of the anatomy (A) resulting from movement of the anatomy (A) at step 112.

The several types of anatomy (A) and types of movements have been described above. The states can be captured for any of these anatomy (A) examples. Also, the states can be captured from any of type of movement described, in part or in whole. Furthermore, as described, the system 10 can capture states of the anatomy (A) using various systems, techniques and/or modalities.

In one example, the states of the anatomy (A) are obtained by tracking the states of the patient trackers 54, 56, which may be coupled to the patient 12 internally (fixed to bone) or externally. For instance, the states of the anatomy (A) can be captured by the navigation system 32, with or without trackers, and by using any one or more of: optical, ultrasound, machine or computer vision, radio frequency, and/or electromagnetic tracking. Additionally, or alternatively, the sensing system(s) 84 can be used. Furthermore, kinematic data can be utilized from either the manipulator 14 or the anatomical manipulator 140, if applicable.

The one or more controllers 60, 36, 26 can also obtain data from the GUI to facilitate capturing states of the anatomy (A) during movement thereof. In other words, the one or more controllers 60, 36, 26 may obtain data related to whether prescribed or recommended movements have been performed and/or may analyze states of the moveable indicator 134 relative to the target indicator 132 in order to make determinations about how the anatomy (A) was moved.

An anatomical manipulator tracker 150 can also be coupled to the anatomical manipulator 140, as shown in FIG. 11, for example, to determine states of the anatomical manipulator 140, or any component thereof, in the localizer coordinate system LCLZ. In such instances, the patient trackers 54, 56 may or may not be utilized. The one or more controllers 60, 36, 26 can analyze the states of the anatomical manipulator tracker 150 to determine or infer the states of the anatomy (A) that is moved by the anatomical manipulator 140. In other instances, the anatomical manipulator tracker 150 is tracker-less but the patient trackers 54, 56 are utilized. Here, the one or more controllers 60, 36, 26 can track the patient tracker 54, 56 to determine states of the anatomy (A), and optionally compare the tracked data to the kinematic data from the anatomical manipulator 140 for validation of movement.

Captured state data from any of these techniques or sources can be utilized and combined. The states of the anatomy (A) can be logged or stored in a non-transitory memory for access by the one or more controllers 60, 36, 26.

### 4. Determine Operative Parameters of Anatomy

With reference to FIG. 7, the method 100 includes the step 152 of the one or more controllers 60, 36, 26 determining operative parameters OP of the anatomy (A). One objective of defining the operative parameters OP is to provide patient-specific guidance for the placement of the manipulator 14 relative to the anatomy (A), as will be described in the next section.

As used herein “operative parameter” defines parameters of how the anatomy (A) moves or is capable of moving in space, and more particularly, during the surgical procedure. FIG. 5 provides a graphical illustration of an example of operative parameters OP shown relative to the anatomy (A), e.g., a patient’s knee. The operative parameters OP can consider external or internal portions of the anatomy (A), such as the surgical site SS. In one implementation, the operative parameters OP include a reachable workspace which defines the limits of where the anatomy (A) is capable of reaching, considering surgical procedure limitations such as restraints on patient movement during the procedure. The operative parameters OP, in one example, include an operative boundary or envelope. The operative parameters OP can define a space, which can be a volume or area. The volume or area can be an open or closed geometry. Depending on the patient and procedure, the operative parameters OP can define a Cartesian, cylindrical, spherical, and/or articulated space or any other partial version thereof. The operative parameters OP can be an actual or physical space and/or data defining this space. The operative parameters OP can be visualized by the GUI or data that is hidden to the user.

The operative parameters OP can be defined by, derived by, and/or augmented from data of various sources. In one implementation, as shown at box 154 in FIG. 7, the operative parameters OP are based, in part or in whole, on the states of the anatomy (A) that were captured during movement in step 148. The captured states, which could be captured using any source and/or technique described, can be transformed into the operative parameters OP. The operative parameters OP can define parameters of the movement of the anatomy (A) as described above, e.g., discrete and/or continuous poses or positions, physical limits, and the like. In one example, the operative parameters OP can be defined by a space swept out by the anatomy (A) during movement in step 148. In another instance, the operative parameters OP can be defined by the total space swept in all possible anatomy (A) motions. The possible motions can be predictively determined by the one or more controllers 60, 36, 26, even in instances where the anatomy (A) was moved in less than all possible motions at step 148. In another implementation, the operative parameters OP can be derived from discrete poses that were captured during movement in step 148. The one or more controllers 60, 36, 26 can envelope the discrete poses to determine a boundary of movement of the anatomy (A). In another example, the operative parameters OP can be used to derive or identify certain poses or landmarks of the anatomy (A). For instance, the operative parameters OP can store a coordinate of the knee center, a location of where the tilt of the knee is at 0 degrees, or where the flexion of the knee is at 105 degrees. Other landmarks and poses are contemplated.

At box 156, the operative parameters OP can be defined by, derived by, and/or augmented from patient data. The patient data can include any physical characteristics of the patient 12, which can inform the one or more controllers 60, 36, 26 to understand anatomy (A) movement. The physical characteristics can include, but are not limited to: patient height, weight, BMI, sex, age, medical or clinical history including injury and disorder history, joint flexibility or laxity, joint range of motion, varus/valgus knee alignment, bone mineral density, and the like. In another implementation, the patient data can be patient imaging data of the anatomy (A), such as CT, X-Ray, Fluoroscopy image data, or the like. For instance, imaging data may reveal the size of the surgical site SS, e.g., the volume of bone that needs to be removed during the procedure. The patient data can also include patient wearable sensor data that could be obtained from the patient having worn wearable sensors on the anatomy (A). For instance, wearable leg or knee tracker data may provide information about the kinematics of the leg or knee, the range of motion, forces applied to the knee during walking, and/or gait information related to patient motion.

At box 158, the operative parameters OP can be defined by, derived by, and/or augmented from surgical plan data. The surgical plan data can include any actual or planned details about the patient 12 and/or manipulator 14, which can inform the one or more controllers 60, 36, 26 to understand anatomy (A) movement. The surgical plan data can be any detail involved with the surgery of the subject anatomy (A), including those described at box 110. The surgical plan data can be determined by a surgeon, can be a specified or predetermined set of parameters for given situation, condition, or procedure, and/or the surgical plan data can be patient-specific, or based on statistical data as is described below. The surgical plan data can include the type of procedure, the type of implant, the approach (surgical access direction) of the procedure, parameters for different steps of the procedure, the types and geometries of tools of the procedure and when such tools are planned to be used or changed during the procedure, parameters of the anatomy, including planned treatment region, cut plane poses, target axes, resection volumes, or the like. The surgical plan data can include a 3D model of the anatomy (A) that is derived from patient imaging data. The surgical plan data can include gap or ligament balancing plans or tools. The surgical plan can include a specified patient outcome, such as a desired range of motion of the anatomy (A). The surgical plan data can include preferred anatomical poses during one or more steps of the surgery. The surgical plan data can include the virtual boundaries generated by the boundary generator 66 where such virtual boundaries are associated with the anatomy (A). The surgical plan data can include how many virtual boundaries, the location of these virtual boundaries, which may inform or limit certain poses of the anatomy (A). The surgical plan data can include the tool paths for the anatomy (A) that are generated by the path generator 68, which may inform or limit certain poses of the anatomy (A). The surgical plan data can include registration techniques, such as using the pointer P to register the anatomy (A), which may inform or limit certain poses of the anatomy (A). Operative parameters OP can also be derived from limitations or preferences related to the anatomy trackers 54, 56 and their relationship to the navigation system 32. For instance, the operative parameters OP could define line-of-sight conditions related to visibility of the anatomy trackers 54, 56 relative to the localizer 44. This data could define the range of motion of the anatomy (A) or trackers 54, 56 that is preferred to optimize visibility and/or limit the anatomy (A) or trackers 54, 56 from assuming certain poses that may obstruct tracker visibility. Any of the described surgical plan data examples can be specific to any procedure or any single step of a procedure. Other types of surgical plan data are contemplated.

At box 160, the operative parameters OP can be defined by, derived by, and/or augmented from statistical data. Here statistical data means non-patient specific data that is collected from a sample size of other patients and/or procedures. The statistical data can be a statistical version of any of the data described above, e.g., patient movement, patient data, surgical plan data, and the like. The one or more controllers 60, 36, 26 can use this statistical data to predict characteristics or movement of the subject anatomy (A). In some instances, the captured states and/or actual patient data can be augmented by statistical data. For instance, the one or more controllers 60, 36, 26 can predict operative parameters OP of the subject anatomy (A) using statistical data related other patients having similar physical characteristics and who have had a similar surgical plan as the subject anatomy (A). In another example, if certain discrete poses of the subject anatomy (A) were captured during movement of the anatomy (A) at step 148, the one or more controllers 60, 36, 26 may “fill in” the range of motion of the subject anatomy (A) based on statistical data related to statistically similar anatomies and their respective movements.

The operative parameters OP can be derived using any combination of the sources described herein, or using other sources not specifically defined herein. Also, the one or more controllers 60, 36, 26 can obtain the operative parameters OP from any suitable source and in any suitable manner. For instance, the operative parameters OP can be stored locally on a non-transitory memory that is accessible to the one or more controllers 60, 36, 26. The memory can be located anywhere, including on the manipulator 14, on the cart 17, on the navigation system 32, on a remote server or cloud, or using a memory drive that is manually provided to the one or more controllers 60, 36, 26. The operative parameters OP can be transmitted to the one or more controllers 60, 36, 26 using a wired or wireless connection. The operative parameters OP can be obtained pre-operatively and/or intraoperatively. In some instances, the operative parameters OP can be derived from artificial intelligence and/or machine learning algorithms which take into consideration post-operative surgical data.

### 5. Determining Desired State of Manipulator

With continued reference to FIG. 7, the method 100 includes the step 170 of the one or more controllers 60, 36, 26 determining the desired state DS of the manipulator 14. The several types of desired states DS for several types of manipulators 14 have been described above and are not repeated herein for simplicity. Some examples of the desired state DS are shown in FIGS. 13 and 14, as displayed on the GUI. The desired state (DS) can be determined using any information described herein, whether used individually or in combination.

In one implementation, the desired state DS defines placement of the cart 17 and/or base 16 of the manipulator 14 relative to the anatomy (A). In one example, as shown in FIG. 13, the desired state DS can be a location of the cart 17 and/or base 16 of the manipulator 14 shown on the GUI relative to the anatomy (A). However, the techniques described herein can also be used to determine the desired state DS relative to any surgical object other than the anatomy (A).

When placed relative to the anatomy (A), the desired state DS can be an optimal state of the manipulator 14 relative to the anatomy (A) where the optimal state accounts for movement of the anatomy (A). Hence, at box 172, the one or more controllers 60, 36, 26 can determine the desired state DS of the manipulator 14 based on an evaluation involving the workspace parameters WP of the manipulator 14 (as obtained at step 102) and the operative parameters OP of the anatomy (A) (as determined at step 152). Here, the one or more controllers 60, 36, 26 can analyze any combination of the described workspace parameters WP and operative parameters OP, and associated data, to determine the desired state DS. Based on the operative parameters OP, the one or more controllers 60, 36, 26 may determine a location, outline, or zone in which to place the cart 17 and/or base 16 of the manipulator 14. Here, the placement of the cart 17 and/or base 16 of the manipulator 14 to the desired state DS can be specifically designed such that the manipulator 14 can reach the anatomy (A) for any identified or possible poses of the anatomy (A) as defined by the operative parameters (OP). The details of the workspace parameters WP and the operative parameters OP may be hidden from the user who guides placement of the manipulator 14.

In another implementation, as shown at box 174, the desired state DS can be specifically defined based on a geometric evaluation involving boundaries (e.g., volumes/areas) derived from the workspace parameters WP and the operative parameters OP. For instance, as shown in FIG. 14, the one or more controllers 60, 36, 26 may define a boundary (shown as a circle) of the workspace parameters (WP) of the manipulator 14 and a boundary (shown as an irregular shape) of the operative parameters OP of the anatomy (A). Here, the one or more controllers 60, 36, 26 can determine the desired state DS by determining how much of the boundary of the operative parameters OP are encompassed by the boundary of the workspace parameters WP. In one example, the desired state DS can be located at any position in which the workspace parameters WP boundary encompasses the operative parameters OP boundary. For example, as shown in FIG. 14, the circle of the workspace parameters WP boundary fully encompasses the operative parameters OP boundary. In another implementation, the desired state DS can be located at any position in which the workspace parameters WP boundary partially encompasses the operative parameters OP boundary. For example, the circle of the workspace parameters WP boundary in FIG. 14 may partially encompass the operative parameters OP boundary. Here, the one or more controllers 60, 36, 26 may determine that a partial encompassing may be appropriate based on certain identified limitations of on full range of motion movement of the anatomy (A) and/or manipulator 14.

The one or more controllers 60, 36, 26 may further define or fine-tune the desired state DS based on the current state CS of the manipulator 14, as shown at box 176. For instance, the desired state DS may consider a path of travel of the manipulator 14 from the current state CS to the desired state DS. The current orientation or direction of the manipulator 14 can also be considered. This evaluation can be performed to optimize the intended path to the desired state DS and/or to minimize the work or difficulty a user may experience in moving the manipulator 14 to the desired state DS. For instance, suppose the current state CS of the manipulator 14 is parallel to the surgical table (as shown in FIG. 13) and the one or more controllers 60, 36, 26 have already determined a desired state DS based on evaluation of the workspace parameters WP and the operative parameters OP. The one or more controllers 60, 36, 26 may determine that a turning radius of the manipulator 14 may be difficult to perform based on evaluation of the current state CS to the desired state DS. In this example, the one or more controllers 60, 36, 26 may slightly expand the zone of the desired state DS or slightly translate the location of the desired state DS to enable the manipulator 14 to accomplish the difficult turn, while still maintaining the desirability of the state based on the evaluation of the workspace parameters WP and the operative parameters OP. In another example, the one or more controllers 60, 36, 26 can identify obstacles in the operating room using any localization or sensing systems described. The desired state DS can be similarly modified to avoid these obstacles.

### 6. Guided Placement of Manipulator to Desired State

Having defined the desired state DS at step 170, the one or more controllers 60, 36, 26 implement guided placement of the manipulator 14 from the current state CS to the desired state DS at step 178 in FIG. 7. As described above, placement of the manipulator 14 to the desired state DS could involve: placement of the cart 17 and/or base 16 of the manipulator 14 to a certain location, region, or zone, placement of the joints (J) and link 18 of the manipulator 14 arm in a desired pose, and/or any other type of placement described, which may depend on the type of manipulator 14. This guidance can be implemented in different manners, which are described below. Any of these implementations can be combined, in part, or in whole.

### i. Manual Guidance

In one implementation, and as shown at box 181, the one or more controllers 60, 36, 26 provide guidance to the user to manually place the manipulator 14 to the desired state DS. In these examples, the user can manually push/pull and steer the manipulator 14 to the desired state DS and/or adjust the pose of the manipulator 14 but does so based on some controller-guided involvement. Manual guidance can be implemented using various techniques. Any of these examples could be combined.

The one or more controllers 60, 36, 26 can provide feedback in the operating room environment to guide the user on manually placing the manipulator 14. For instance, the one or more controllers 60, 36, 26 can implement audible feedback and instructions from a speaker that is coupled to the cart 17 and/or navigation system 32 or any other device in the operating room, such as a head-mounted device. The audible instructions are derived from comparison of the current state CS to the desired state DS. The audible instructions may alert the user using specific audible commands and distances, for example, to “push the manipulator forward 3 feet”, “turn the manipulator 45 degrees”, or “park the manipulator”. The audible directions can persist until the one or more controllers 60, 36, 26 determine that the desired state DS has been reached.

In another implementation, the one or more controllers 60, 36, 26 can implement visual feedback and instructions on a display device, e.g., the navigation displays 38, a display on the cart 17, a head-mounted device, or the like. The visual instructions are derived from comparison of the current state CS to the desired state DS. The visual instructions may alert the user using specific visual indicators or information, for example, by displaying an arrow to instruct a direction in which to move the manipulator 14, displaying written instructions “turn the manipulator perpendicular to the surgical table”.

In another example, a head-mounted device could be worn by the user to provide any version of the described audible or visual feedback. For instance, the head-mounted device could project the desired state DS in the operating room using augmented reality or mixed reality. The user can place the manipulator 14 to the desired state DS and the head-mounted device could provide feedback once the desired state DS has been reached. In one example, the head-mounted device could have a forward-facing camera to detect presence of the base 16 of the manipulator 14 and/or cart 17 in the desired state DS and provide confirmation feedback to the user.

In another implementation, the one or more controllers 60, 36, 26 can implement haptic feedback. Haptic feedback can be any feedback that is physically perceptible by user feel or touch. In one example, the user could wear a haptic device, such as a haptic bracelet that could vibrate upon detection of the manipulator 14 in the desired state DS and/or could vibrate to provide directions to guide the user. For instance, the bracelet could have different vibrating zones around the wrist, and the left zone could vibrate to guide the user to move the manipulator 14 to the left, and so on. In another implementation, haptic feedback can be provided to manual steering controls of the cart 17.

In another example, the cart 17 and/or base 16 of the manipulator 14 can be equipped with a placement control system 180, for example, as shown in FIG. 4. The placement control system 180 assists in guidance in placement of the manipulator 14. The placement control system 180 can include a controller 182, which can include any part of, or communicate with, the one or more controllers 60, 36, 26 or could be a separate controller altogether. Communication could be wirelessly implemented between the various controllers. The cart 17 has wheels 184 to enable the cart 17 to move along the surface of the floor. Some or all of the wheels 184 could be actively driveable and steerable wheels. The placement control system 180 could include a drive system 186 that is coupled to the controller(s) 60, 36, 26, 180 and the driven wheels 184 to drive, e.g., rotate, the wheels 184 forward or backwards. The controller(s) 60, 36, 26, 182 can control the drive system 186 to change the velocity, acceleration or braking of the cart 17. The placement control system 180 further includes a steering system 188 that is coupled to the controller(s) 60, 36, 26, 182 and to the wheels 184 to steer the wheels 184. The controller(s) 60, 36, 26, 182 can control the steering system 188 to change the direction, orientation, and path of the cart 17. One example of a steering system that could be implemented to control the direction of the cart can be like that described in US Pat. No. 10,231,792, entitled “Carriage for Portable Surgical Robot”, the contents of which are hereby incorporated by reference in their entirety.

The placement control system 180 can be used to guide the user on manually placing the manipulator 14 to the desired state DS. For instance, the placement control system 180 can provide haptic feedback to the user during user-initiated movement of the cart 17. In one example, the controller(s) 60, 36, 26, 182 are aware of the current and desired states, CS, DS of the cart 17 using any described technology and methodology. Knowing this relationship, the controller(s) 60, 36, 26, 182 can develop one or more haptic boundaries to help guide the user to move the base 16 and/or cart 17 to the desired state DS.

In one example, as shown in FIG. 13, the haptic boundary is a haptic path HP of travel that the user should follow to move the base 16 and/or cart 17 to the desired state. The controller(s) 60, 36, 26, 182 can develop this haptic path HP based on the data from step 170, which involves evaluation of the workspace parameters WP, operative parameters OP, and the like. As the user moves the base 16 and/or cart 17 manually, the placement control system 180 can perform actions with the steering and/or drive systems 186, 188 to provide reactive feedback when certain conditions are identified related to the haptic path HP. For instance, if the user manually pushes the cart off the haptic path HP, the placement control system 180 can control the steering or drive systems 186, 188 to restrict turning of the wheels 184 from deviating from the haptic path HP, turn the wheels 184 to get back onto the haptic path HP, and/or to vibrate the wheels 184 or steering controls to haptically inform the user of such deviations. Deviation from the haptic path HP can be strictly enforced to only allow movement on the path. Alternatively, the haptic path HP can define limits that permit the user to deviate from the path, but only by a certain amount, e.g., defining a two-meter haptic lane that is parallel with and centered to the path. In another instance, the placement control system 180 can control the steering or drive systems 186, 188 to stop or slow the wheels 184 if the user deviates from the path or reaches the desired state DS. The placement control system 180 can also determine a predetermined velocity limit that the base 16 and/or cart 17 should not exceed and control the drive system 186 to reduce the velocity in response to the user manually pushing the base 16 and/or cart 17 beyond the limit. Hence, in these examples, the user remains in control of placing the manipulator 14, but the placement control system 180 can provide guided assistance as needed.

Additionally, or alternatively, the haptic boundary could be implemented by the controller(s) 60, 36, 26, 182 providing a haptic zone HZ, for example, as shown in FIG. 13. The haptic zone HZ can be virtually located near, adjacent to, or around the local region of the desired state DS. Here, the user may have full autonomy to manually push the cart 17 along any path they wish, until the base 16 and/or cart 17 breaches the haptic zone HZ or reaches the haptic zone HZ, e.g., within 1 meter. In one implementation, the navigation system 32 can associate a virtual object (e.g., a virtual outline) with the base 16 and/or cart 17 to determine if the virtual object reaches or breaches the haptic zone HZ. Once the base 16 and/or cart 17 breaches or reaches this haptic zone HZ, the placement control system 180 could automatically trigger haptic feedback through the wheels 184, as described above. For instance, the placement control system 180 could steer, drive, stop, and/or vibrate steering control or the wheels 184 to inform the user of the boundary of the haptic zone HZ.

### ii. Automated Guidance

In another implementation, and as shown at box 185, the one or more controllers 60, 36, 26, 182 provide automated guidance to place the manipulator 14 or cart 17 to the desired state DS. In these examples, guidance can be fully automated or semi-automated. When fully automated, the user need not be involved and the one or more controllers 60, 36, 26, 182 can perform all actions needed for the manipulator 14 or cart 17 to reach the desired state DS. When semi-automated, the user may be involved with override/emergency stop actions. For instance, the user may need to hold down a switch throughout the duration of semi-automated movement of the manipulator 14 or cart 17. If the switch is released, the automated actions will stop. This switch may be located on the handle of the cart 17. Semi-automated control may also involve the one or more controllers 60, 36, 26, 182 performing some actions, while leaving other actions to the user. For instance, the placement control system 180 can provide automated steering while leaving the pushing of the base 16/cart 17 to the user. Alternatively, the placement control system 180 can provide automated driving of the wheels 184 while leaving the steering of the cart 17 to user direction. Other types of semi-automated control are envisioned.

For automated or semi-automated guidance, the controller(s) 60, 36, 26, 182 can develop an automated path AP of travel that placement control system 180 can follow to automatically move the manipulator 14, base 16, or cart 17 from the current state CS to the desired state DS. During automated movement, the placement control system 180 can drive and steer the cart 17 with the steering or drive systems 186, 188. Along the automated path AP, the placement control system 180 can dynamically change the automated path AP to avoid detected obstacles. Automated movement may occur according to a predefined velocity plan or velocity limit for the wheels 184. Once the desired state DS has been reached, the placement control system 180 can automatically stop the wheels 184 and the manipulator 14 or cart 17. Automated guidance may also be implemented by the user pushing the cart 17 manually until the haptic zone HZ is reached or breached. Once this occurs, the placement control system 180 can take over control and auto-park the manipulator 14 to the desired state DS. This can further involve the controller(s) 60, 36, 26, 182 placing the manipulator 14 arm in the desired pose once parked. Any of the manual and automated techniques can be combined in part, or in whole.

### iii. GUI Guidance

Whether the cart 17 and/or manipulator 14 are controlled in a manual, semi-automated, or fully automated manner, the placement to the desired state DS can be further implemented by using the GUI, as shown at box 187 in FIG. 7 and as depicted in the examples of FIGS. 13 and 14. In the examples shown, the GUI can enable the user to visually see an actual or graphical representation of any of the following: the operating room, the cart 17, 17′ or manipulator 14, 14′ at the current state CS, the patient 12, 12′ or anatomy A, A′, and a graphical representation of the desired state DS. The GUI can also show actual/graphical representations of real-time movement of the cart 17, 17′ and/or manipulator 14, 14′ which correspond to movement of the cart 17 and/or manipulator 14 in the operating room. Furthermore, the above-described haptic boundaries, e.g., haptic paths HP or haptic zones HZ, could be shown on the GUI to inform the user that these features are active and to help the user understand the planned guidance provided by these boundaries.

The graphical representations can be similar to or different from those shown in FIGS. 13 and 14. For instance, the operating room representation may not include all objects in the operating room. The base 16, cart 17 and/or manipulator 14 could be shown generically. Furthermore, the images in FIGS. 13 and 14 are shown from an overhead layout view of the operating room, which may or may not be to scale. Alternatively, any of the described imagery could be shown in 3-dimenisons.

The desired state DS is shown in FIG. 13 as a box or zone but could be shown any other way. For example, the desired state DS could be shown as a 3D outline, shadow, or silhouette of the base 16, cart 17 and/or manipulator 14. In another example, as shown in FIG. 14, the desired state DS may not be expressly shown. Instead, the desired state DS can be reached based on a geometric comparison on the GUI between graphical representations of the workspace parameters WP′ of the manipulator 14 and the operative parameters OP′ of the anatomy A, A′. For instance, in the example shown, a circle geometrically represents the workspace parameters WP′ of the manipulator 14 and an irregular shape geometrically represents the operative parameters OP′ of the anatomy A, A′. The graphical representation of the workspace parameters WP′ can be graphically fixed to the cart 17, 17′ or manipulator 14, 14′ and will follow the cart 17, 17′ or manipulator 14, 14′ during movement thereof. Similarly, the graphical representation of the operative parameters OP′ can be graphically fixed to the anatomy A, A′ and may change location on the GUI if the anatomy A, A′ were to move. In one example, the desired state DS can be any location of the cart 17, 17′ and manipulator 14, 14′ in which the graphical representation of the workspace parameters WP′ fully encompasses the graphical representation of the operative parameters OP′. For example, in FIG. 14, the circle encompasses the irregular shape. In other examples, as described, there could be partial overlap between these graphical representations.

To use the GUI for manual or semi-automated guidance, the user can manually move the base 16, cart 17 and/or manipulator 14 using any of the guidance techniques described while referring to the display screen 38 for guidance. In FIG. 13, user can see the relationship between the current state CS and the desired state DS on the GUI and can continue to move the base 16, cart 17 and/or manipulator 14 until the desired state DS is reached. In FIG. 14, user can see the relationship between the graphical representations of the workspace parameters WP′ and the operative parameters OP′ on the GUI and can continue to move the base 16, cart 17 and/or manipulator 14 until the desired state DS is achieved. In either example, the GUI can provide any type of visual and/or audible feedback to guide the user to the desired state DS during movement of the base 16, cart 17 and/or manipulator 14. For instance, the GUI may provide text or speech indicating to “Move the robot forward 100 mm”. Once the desired state DS is achieved, the GUI can provide a state confirmation indicator that is configured to be triggered in response to the cart 17, 17′ and manipulator 14, 14′ reaching the desired state DS. This state confirmation indicator could be changing the color of the desired state DS, e.g., box in FIG. 13, to green and/or providing a confirmation speech or chime.

For fully automated guidance, the user can refer to the display screen 38 as the placement control system 180 moves the base 16, cart 17 and/or manipulator 14 to the desired state DS in an automated manner. The user can refer to the GUI to confirm that automated guidance is being performed as planned, e.g., the cart 17 is being moved along the planned path of travel.

Furthermore, any of the above GUI guidance techniques can be implemented on a head-mounted device with a transparent lens directly in front of the eyes of the user. In these scenarios, the patient 12 and the current state CS of the base 16, cart 17 and/or manipulator 14 may not need to be shown on the GUI as they could be clearly visible through a transparent lens of the head-mounted device. The desired state DS, and/or the graphical representations of the workspace parameters WP′ and the operative parameters OP′ can be shown on the lens of the head-mounted device using augmented or mixed reality. For instance, the desired state DS could be a box outline that extends along the floor, or a 3D silhouette of the base 16, cart 17 and/or manipulator 14 at the desired state DS. The pose and/or size of the desired state DS and/or the graphical representations of the workspace parameters WP′ and the operative parameters OP′ will change according to the location and perspective of the user. The user wearing the head-mounted device could manually place the base 16, cart 17 and/or manipulator 14 using guidance from the head-mounted device. Alternatively, the user could utilize the head-mounted device to observe automated placement of the base 16, cart 17 and/or manipulator 14. Guidance using the GUI is contemplated using any of these techniques, in part, or in whole.

### 7. Dynamic Adjustment for Changed Conditions

Referring to FIG. 7, the method 100 can further comprise the step 190 of detecting conditions or changes that may affect placement of the base 16, cart 17 and/or manipulator 14. These conditions or changes can occur at any time or during any of the described steps of method 100. For example, the conditions or changes may occur before, during, or after placement of the manipulator to the desired state DS. These conditions or changes may be unexpected, planned or may result from input by the surgeon.

Described above, the one or more controllers 60, 36, 26 can obtain or determine the workspace parameters WP of the manipulator 14 and the operative parameters OP of the anatomy (A) using numerous implementations. For simplicity in description, these parameters and their sources are not repeated herein. If the one or more controllers 60, 36, 26 detect a deviation from prior obtained data or from prior determinations related to these parameters WP, OP (shown at boxes 192 and 194, respectively) the controllers trigger a response. The response can be repeating, revaluating, or reobtaining data from any one or more of the following steps: step 102 obtaining workspace parameters WP of the manipulator 14; step 112 moving the anatomy (A); step 138 capturing states of the anatomy (A) during movement; step 152 determining operative parameters OP of the anatomy (A); step 170 determining the desired state DS of the manipulator 14; and/or step 178 guiding placement of the manipulator 14 to the desired state DS.

In one example, the parameters WP, OP include details for different surgical procedure steps related to the anatomy (A) relative to the manipulator 14. The one or more controllers 60, 36, 26 can automatically detect, or receive user input of, start or completion of any given step of the procedure. The manipulator 14 may be placed in the desired state DS during one step of the procedure according to the described techniques. After completion of the one step, and before start of the subsequent step, the one or more controllers 60, 36, 26 may automatically determine a new desired state DS for the manipulator 14. This evaluation may further include a desired state of the anatomy (A). Guidance can then be implemented using any described technique.

In another example, during the procedure, the one or more controllers 60, 36, 26 can determine an error condition in which the localizer 44 is unable to track any one or more of the manipulator trackers 52A, 52B and/or the anatomy trackers 54, 56. The controllers can then display, on the display device 38 or GUI, an error indicator configured to alert of the error condition and/or to provide guidance to resolve the error condition. Resolution may involve the one or more controllers 60, 36, 26 automatically determining a new desired state DS for the manipulator 14 and/or a desired state of the anatomy (A).

Other examples of changed conditions that may result in a new desired state DS of the manipulator 14, base 16, and/or cart 17 or a new desired state of the anatomy (A) may include but are not limited to: manipulator 14 calibration or accuracy errors or changes; changes to the surgical plan; errors related to the trackers, such as lost registration or tracking, unexpected or planned changes in the position of the manipulator 14 or anatomy (A), and the like. Any change or condition arising from the described parameters WP, OP is contemplated.

### D. Example Advantages and Technical Solutions

The techniques described herein enable guidance of proper and/or optimal placement of the manipulator 14, base 16, and/or cart 17 and particularly, relative to the patient 12 in the operating room thereby avoiding interruption to the surgical procedure. Other advantages of the techniques described herein include but are not limited to: (1) reducing human error in placement of the manipulator 14, base 16, and/or cart 17; (2) defining a desired state DS of the manipulator 14, base 16, and/or cart 17 that is patient-specific, procedure-specific, step-of-procedure specific, and/or manipulator-specific, or any combinations thereof (3) defining a desired state DS of the manipulator 14, base 16, and/or cart 17 that is finely tuned to the patient anatomy (A) and variables associated with the anatomy (A), such as but not limited to: surgical site SS parameters; anatomical joint parameters; joint range of motion; joint flexibility; joint laxity; height, length and/or width of limb(s); location of trackers on the anatomy (A); patient size; volume of material removed for anatomy (A); and the like (4) defining a desired state DS of the manipulator 14, base 16, and/or cart 17 that is finely tuned to the manipulator 14 and variables associated with the manipulator 14, such as but not limited to: manipulator parameters; manipulator range-of-motion; manipulator joint limits; height of the manipulator 14 in moving and parked states; height of the manipulator 14 relative to the patient 12; manipulator singularities; the degrees-of-freedom for which the manipulator 14 will be allowed or constrained during the procedure, or during steps of the procedure; virtual boundaries to which the manipulator 14 will be constrained during the procedure, or during steps of the procedure; the tool path along which the manipulator 14 will move the tool 20 the during the procedure, or during steps of the procedure; the tools 20 that will be used during the procedure or changing of tools that occur the manipulator during the procedure, or during steps of the procedure; (5) defining a desired state DS of the manipulator 14, base 16, and/or cart 17 that is finely tuned to the surgical procedure and variables associated with the surgical procedure, such as but not limited to: the surgical plan; changes to the surgical plan; planned poses of the manipulator 14 throughout the procedure; planned poses of the patient throughout the procedure; the location of the surgical site SS (e.g., left or right knee); the location of the surgical table; the location or planned location of the surgeon or staff relative to the patient 12 and/or manipulator 14; the location or change of state of the navigation system 32, localizer 44 or trackers in the operating room; and the like (6) defining a desired state DS of the manipulator 14 that accounts for how the manipulator 14 can effectively, efficiently, and without interference reach the desired state DS (7) defining a desired state DS of the manipulator 14 that can be modified by computer-implemented detection or predicted detection of patient-specific, procedure-specific, step-of-procedure specific, and/or manipulator-specific conditions that may prompt changing of the desired state DS (8) enabling manual or automated, or combined manual/automated placement of the manipulator 14 to the desired state DS, and optionally doing so using visual, audible and haptic feedback implemented by haptic boundaries, such as haptic paths of travel or haptic zones (9) enabling automated movement of a patient’s limb to provide input into guiding placement of the manipulator based on the outcome of the patient’s movement. Other advantages will be understood from the above description.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A surgical system comprising:
a robotic manipulator;
a localizer configured to track the robotic manipulator and an anatomy of a patient; and one or more controllers coupled to the localizer and being configured to:
obtain workspace parameters of the robotic manipulator;
capture, from the localizer, a current state of the robotic manipulator relative to the anatomy;
capture, from the localizer, states of the anatomy in response to movement of the anatomy according to a prescribed manner or a predetermined manner;
determine operative parameters of the anatomy based on the captured states of the anatomy;
compare the workspace parameters to the operative parameters to determine a desired state for the robotic manipulator relative to the anatomy, whereby in the desired state, the workspace parameters of the robotic manipulator have an acceptable relationship with respect to the operative parameters of the anatomy; and
guide placement of the robotic manipulator from the current state to the desired state.

2. The surgical system of claim 1, wherein the anatomy comprises an anatomical joint, and the prescribed manner or the predetermined manner includes one or more of: flexing the anatomical joint; extending the anatomical joint; tilting the anatomical joint; and rotating the anatomical joint.

3. The surgical system of claim 1, further comprising a display device, and wherein the one or more controllers are configured to:
prompt a user, on the display device, to manually move the anatomy according to the prescribed manner; and
capture, from the localizer, the states of the anatomy in response to movement of the anatomy according to the prescribed manner.

4. The surgical system of claim 1, further comprising an anatomical manipulator being configured to support and move the anatomy, wherein the one or more controllers are coupled to the anatomical manipulator and are configured to:
command the anatomical manipulator to autonomously or semi-autonomously move the anatomy according to the predetermined manner; and
capture the states of the anatomical manipulator in response to autonomous or semi-autonomous movement of the anatomy by the anatomical manipulator according to the predetermined manner.

5. The surgical system of claim 1, wherein the one or more controllers are configured to determine the operative parameters of the anatomy based on any one or more of:
the states of the anatomy being captured at physical range of motion limits of the anatomy;
the states of the anatomy being captured during continuous motion of the anatomy;
the states of the anatomy being captured at one or more discrete positions within physical range of motion limits of the anatomy; and
augmentation of the captured states with one or more of: patient data, surgical plan data, and statistical data.

6. The surgical system of claim 1, further comprising a display device, and wherein the one or more controllers are configured to guide movement of the anatomy according to the prescribed manner by further being configured to display, on the display device, a target indicator comprising one or both of:
a target position at which to place the anatomy; and
a target range within which to place the anatomy.

7. The surgical system of claim 6, wherein the one or more controllers are configured to guide movement of the anatomy according to the prescribed manner by further being configured to display, on the display device:
a moveable indicator that is configured to move in response to movement of the anatomy and to move relative to one or both of:
the target position to provide guidance on relative positioning between the anatomy and the target position; and
the target range to provide guidance on relative positioning between the anatomy and the target range; and
a target confirmation indicator that is configured to be displayed in response to the moveable indicator being located at the target position and/or within the target range.

8. The surgical system claim 1, wherein the one or more controllers are configured to guide placement of the robotic manipulator from the current state to the desired state by being configured to display, on a display device:
a representation of the robotic manipulator at the current state;
a representation of the anatomy;
a graphical representation of the desired state of the robotic manipulator;
movement of the representation of the robotic manipulator from the current state to the desired state; and
a state confirmation indicator that is configured to be displayed in response to the representation of the robotic manipulator reaching the desired state.

9. The surgical system claim 1, wherein the one or more controllers are configured to guide placement of the robotic manipulator from the current state to the desired state by being configured to display, on a display device:
a graphical representation of the workspace parameters of the robotic manipulator;
a graphical representation of the operative parameters of the anatomy; and
a state confirmation indicator that is configured to be displayed in response to determining presence of the acceptable relationship between the graphical representation of the workspace parameters and the graphical representation of the operative parameters.

10. The surgical system of claim 1, wherein the anatomy is subject to a surgical procedure involving a plurality of steps, and wherein, after successful placement of the robotic manipulator from the current state to the desired state, the one or more controllers are configured to:
identify a change to one or both of: the workspace parameters and the operative parameters;
evaluate the change to determine a second desired state for the robotic manipulator and guide placement of the robotic manipulator from the current state to the second desired state; and/or
evaluate the change to determine a desired pose of the anatomy and guide placement of the anatomy to the desired pose.

11. The surgical system of claim 1, wherein:
the robotic manipulator is configured to be manually moved by a user; and
the one or more controllers guide placement of the robotic manipulator by being configured to display, on a display device, instructions to assist the user to manually move the robotic manipulator from the current state to the desired state.

12. The surgical system of claim 1, wherein the robotic manipulator comprises a robotic arm including a plurality of links and joints, and a cart that is moveable and is configured to support the robotic arm, and wherein the one or more controllers guide placement of the robotic manipulator by further being configured to guide placement of the cart of the robotic manipulator from the current state to the desired state.

13. The surgical system of claim 1, wherein:
the robotic manipulator comprises a robotic arm including a plurality of links and joints, and a manipulator controller that is configured to control the robotic arm; and
the one or more controllers are configured to communicate with the manipulator controller and to further guide placement of the robotic manipulator by being configured to instruct the manipulator controller to autonomously move the robotic arm from the current state to the desired state.

14. The surgical system of claim 1, wherein:
the robotic manipulator comprises a robotic arm including a plurality of links and joints, and a cart being configured to support the robotic arm, the cart comprising a plurality of wheels such that the cart is moveable, and a placement control system comprising a drive system that is configured to drive the wheels, a steering system that is configured to steer the wheels, and a cart controller that is configured to control the drive system and steering system; and
the one or more controllers are configured to communicate with the placement control system and to guide placement of the robotic manipulator to the desired state.

15. The surgical system of claim 14, wherein the placement control system is configured to control the drive system and the steering system to autonomously move the cart from the current state to the desired state.

16. The surgical system of claim 14, wherein the placement control system is configured to provide haptic feedback to guide a user on placement of the robotic manipulator from the current state to the desired state, wherein the haptic feedback is implemented by one or both of:
a haptic path, wherein the placement control system controls the drive system and/or the steering system to provide haptic feedback in response the user manually moving the cart in a manner that interacts with, or deviates from, the haptic path; and
a haptic zone, wherein the placement control system controls the drive system and/or the steering system to provide haptic feedback in response the user manually moving the cart in a manner that interacts with, or deviates from, the haptic zone.

17. The surgical system of claim 1, wherein the one or more controllers are configured obtain the workspace parameters of the robotic manipulator by further being configured to obtain one or more of: a predetermined kinematic model of the robotic manipulator; factory data related to the robotic manipulator; calibration or setup data related to the robotic manipulator; and surgical plan data related to the robotic manipulator.

18. A method of operating a surgical system, the surgical system including a robotic manipulator, a localizer configured to track the robotic manipulator and an anatomy of a patient, and one or more controllers coupled to the localizer, and the method comprising the one or more controllers performing the steps of:
obtaining workspace parameters of the robotic manipulator;
capturing, from the localizer, a current state of the robotic manipulator relative to the anatomy;
capturing, from the localizer, states of the anatomy in response to movement of the anatomy according to a prescribed manner or a predetermined manner;
determining operative parameters of the anatomy based on the captured states of the anatomy;
comparing the workspace parameters to the operative parameters for determining a desired state for the robotic manipulator relative to the anatomy, whereby in the desired state, the workspace parameters of the robotic manipulator have an acceptable relationship with respect to the operative parameters of the anatomy; and
guiding placement of the robotic manipulator from the current state to the desired state.

19. The method of claim 18, wherein the anatomy comprises an anatomical joint, and the prescribed manner or the predetermined manner includes one or more of: flexing the anatomical joint; extending the anatomical joint; tilting the anatomical joint; and rotating the anatomical joint.

20. The method of claim 18, comprising the one or more controllers:
prompting a user, on a display device, to manually move the anatomy according to the prescribed manner; and
capturing, from the localizer, the states of the anatomy in response to movement of the anatomy according to the prescribed manner.

21. The method of claim 18, wherein the surgical system comprises an anatomical manipulator being configured to support and move the anatomy, and the one or more controllers being coupled to the anatomical manipulator, the method comprising the one or more controllers:
commanding the anatomical manipulator to autonomously or semi-autonomously move the anatomy according to the predetermined manner; and
capturing the states of the anatomical manipulator in response to autonomous or semi-autonomous movement of the anatomy by the anatomical manipulator according to the predetermined manner.

22. The method of claim 18, comprising the one or more controllers determining the operative parameters of the anatomy based on any one or more of:
the states of the anatomy being captured at physical range of motion limits of the anatomy;
the states of the anatomy being captured during continuous motion of the anatomy;
the states of the anatomy being captured at one or more discrete positions within physical range of motion limits of the anatomy; and
augmentation of the captured states with one or more of: patient data, surgical plan data, and statistical data.

23. The method of claim 18, comprising the one or more controllers guiding movement of the anatomy according to the prescribed manner by displaying, on a display device, a target indicator comprising one or both of:
a target position at which to place the anatomy; and
a target range within which to place the anatomy.

24. The method of claim 23, comprising the one or more controllers guiding movement of the anatomy according to the prescribed manner by further displaying, on the display device:
a moveable indicator that moves in response to movement of the anatomy and moves relative to one or both of:
the target position for providing guidance on relative positioning between the anatomy and the target position; and
the target range for providing guidance on relative positioning between the anatomy and the target range; and
a target confirmation indicator that is displayed in response to the moveable indicator being located at the target position and/or within the target range.

25. The method of claim 18, comprising the one or more controllers guiding placement of the robotic manipulator from the current state to the desired state by displaying, on a display device:
a representation of the robotic manipulator at the current state;
a representation of the anatomy;
a graphical representation of the desired state of the robotic manipulator;
movement of the representation of the robotic manipulator from the current state to the desired state; and
a state confirmation indicator that is displayed in response to the representation of the robotic manipulator reaching the desired state.

26. The method of claim 18, comprising the one or more controllers guiding placement of the robotic manipulator from the current state to the desired state by displaying, on a display device:
a graphical representation of the workspace parameters of the robotic manipulator;
a graphical representation of the operative parameters of the anatomy; and
a state confirmation indicator that is displayed in response to determining presence of the acceptable relationship between the graphical representation of the workspace parameters and the graphical representation of the operative parameters.

27. The method of claim 18, wherein the anatomy is subject to a surgical procedure involving a plurality of steps, and wherein, after successful placement of the robotic manipulator from the current state to the desired state, the method comprises the one or more controllers:
identifying a change to one or both of: the workspace parameters and the operative parameters;
evaluating the change to determine a second desired state for the robotic manipulator and guiding placement of the robotic manipulator from the current state to the second desired state; and/or
evaluating the change to determine a desired pose of the anatomy and guiding placement of the anatomy to the desired pose.

28. The method of claim 18, wherein the robotic manipulator is manually moved by a user, and comprising the one or more controllers guiding placement of the robotic manipulator by:
displaying, on a display device, instructions for assisting the user to manually move the robotic manipulator from the current state to the desired state.

29. The method of claim 18, wherein the robotic manipulator comprises a robotic arm including a plurality of links and joints, and a cart that is moveable and is configured to support the robotic arm, and wherein guiding placement of the robotic manipulator further comprises the one or more controllers:
guiding placement of the cart of the robotic manipulator from the current state to the desired state.

30. The method of claim 18, wherein the robotic manipulator comprises a robotic arm including a plurality of links and joints, and a manipulator controller coupled to the one or more controllers and being configured to control the robotic arm, and wherein guiding placement of the robotic manipulator further comprises the one or more controllers:
guiding placement of the robotic arm by instructing the manipulator controller to autonomously move the robotic arm from the current state to the desired state.

31. The method of claim 18, wherein the robotic manipulator comprises a robotic arm including a plurality of links and joints, and a cart being configured to support the robotic arm, the cart comprising a plurality of wheels such that the cart is moveable, and a placement control system comprising a drive system that is configured to drive the wheels, a steering system that is configured to steer the wheels, and a cart controller that is configured to control the drive system and steering system, and wherein guiding placement of the robotic manipulator further comprises the one or more controllers:
communicating with the placement control system and for guiding placement of the robotic manipulator to the desired state.

32. The method of claim 31, comprising the one or more controllers instructing the placement control system to control the drive system and the steering system for autonomously moving the cart from the current state to the desired state.

33. The method of claim 31, comprising the one or more controllers instructing the placement control system to provide haptic feedback for guiding a user on placement of the robotic manipulator from the current state to the desired state, wherein the haptic feedback is implemented by one or both of:
a haptic path, whereby the placement control system is controlling the drive system and/or the steering system for providing haptic feedback in response the user manually moving the cart in a manner that interacts with, or deviates from, the haptic path; and
a haptic zone, whereby the placement control system is controlling the drive system and/or the steering system for providing haptic feedback in response the user manually moving the cart in a manner that interacts with, or deviates from, the haptic zone.

34. A non-transitory computer-readable medium configured to be utilized with a localizer configured to track a robotic manipulator and an anatomy of a patient, wherein the non-transitory computer-readable medium comprises instructions, which when executed by one or more processors, are configured to:
obtain workspace parameters of the robotic manipulator;
capture, from the localizer, a current state of the robotic manipulator relative to the anatomy;
capture, from the localizer, states of the anatomy in response to movement of the anatomy according to a prescribed manner or a predetermined manner;
determine operative parameters of the anatomy based on the captured states of the anatomy;
compare the workspace parameters to the operative parameters to determine a desired state for the robotic manipulator relative to the anatomy, whereby in the desired state, the workspace parameters of the robotic manipulator have an acceptable relationship with respect to the operative parameters of the anatomy; and
guide placement of the robotic manipulator from the current state to the desired state.
